(19) United States
(12) Patent Application Publication
(10) Pub. No.: US 2017/0168067 A1
(43) Pub. Date: Jun. 15, 2017

(54) CHROMATIN-ACTIVITY-BASED CHEMOPROTEOMIC (CHAC) METHODS AND SYSTEMS FOR DISEASE MARKER DISCOVERY AND DEVELOPMENT

(71) Applicant: The University of North Carolina at Chapel Hill, Chapel Hill, NC (US)

(72) Inventor: Xian Chen, Chapel Hill (US)

(21) Appl. No.: 15/118,061

(22) PCT Filed: Feb. 11, 2015
(86) PCT No.: PCT/US15/15457

§ 371 Date: Aug. 10, 2016

Related U.S. Application Data
(60) Provisional application No. 61/938,573, filed on Feb. 11, 2014.
(60) Provisional application No. 62/088,302, filed on Dec. 5, 2014.

(51) Int. Cl. G01N 33/68; G01N 33/574; G01N 33/573

(52) U.S. Cl. CPC G01N 33/6875; G01N 33/573; C12Y 201/01043; G01N 33/57488; G01N 2333/91017; G01N 2440/12; G01N 2570/00; G01N 2800/7095

(57) ABSTRACT

Methods for identifying and developing biomarkers based on the characterization of disease-related components of gene-specific chromatin regulatory protein complexes. Chemoprobes that are substrate-competitive and selectively bind enzymatically active enzymes associated with gene-specific chromatin regulatory protein complex can be used to select chromatin complexes associated with a phenotype of interest.

### CROSS-REFERENCE TO RELATED APPLICATIONS

This application claims benefit of U.S. Provisional Patent Application Ser. Nos. 61/938,573, filed Feb. 11, 2014; and 62/088,302, filed Dec. 5, 2014, each of which are herein incorporated by reference in their entireties.

### GRANT STATEMENT

This invention was made with government support under Grant Nos. ROIAI064806 and 1U24CA160035 awarded by the National Institutes of Health. The government has certain rights in the invention.

### TECHNICAL FIELD

This presently disclosed subject matter generally relates to chromatin-activity-based chemoproteomic (ChaC) methods and systems for disease marker development and/or discovery. The presently disclosed subject matter relates to approaches, methods and systems that can generate activity-based interactome data in a physiologically accurate manner and is generically applicable to dissect a variety of phenotypic chromatin architectures that correlate with the regulation of specific genes in the context of a functional protein complexome. The presently disclosed subject matter relates to methods, systems and approaches for identifying and screening for biomarkers that can be assessed for use in disease diagnosis and testing. Further embodiments are described below.

### BACKGROUND

The levels of histone N-terminal post-translational modifications (PTMs), such as methylation, acetylation, phosphorylation, and ubiquitination, determine both the dynamic architecture and the functional state of chromatin, which eventually control chromatin-mediated gene transcription. Meanwhile, the levels of particular histone PTMs are regulated by corresponding histone modifying enzymes, whose activities define the chromatin states that vary in correlating with the cellular phenotypes of health or disease.

Currently, the tasks to characterize chromatin states of genes are primarily accomplished by performing ChIP-seq experiments using antibodies against either particular histone PTMs or known chromatin regulators. Although ChIP-seq represents a non-biased, high-throughput (HT) method that can identify DNA regulatory elements or map the genome-wide binding of given components of chromatin machinery, ChIP data provide static information about chromatin states at the genomic level that are not necessarily functional. In addition, not all antibodies are ChIP-grade, which affects both sensitivity and accuracy of genomic binding mapping. On the other hand, functional characterizations of chromatin protein modifiers/regulators are mostly done one-gene-at-a-time in spite of system complexities. Recently, various HT techniques combining chromatin affinity purification with mass spectrometry (MS) have been developed to identify/profile the components of chromatin proteomic complexes associated with particular genomic regions, performing a systems, unbiased dissection of each epigenetic protein machinery. However, because the antibody-dependent affinity purification pulls down protein complexes based on the abundance of a target/bait protein, a lack of gene specificity can be expected for these complex identities. This therefore adversely affects the accuracy and sensitivity in determining the actual functional states of these chromatin regulators.

As such, there remains a need for methods and systems that can generate activity-based interactome data in a physiologically accurate manner and is generically applicable to dissect a variety of phenotypic chromatin architectures in the context of a functional protein complexome. There also remains a need for improved methods, systems and approaches for identifying and screening for novel biomarkers that can be assessed for use in disease diagnosis and testing.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides methods for identifying a disease-related component of a gene-specific chromatin regulatory protein complex. In some embodiments the disclosed methods comprise providing a sample to be assayed, contacting the sample with a chemoprobe, wherein the chemoprobe is substrate-competitive and selectively binds to an enzymatically active enzyme associated with a gene-specific chromatin regulatory protein complex, and detecting a gene-specific chromatin regulatory protein complex present in the sample using the chemoprobe based on the enzymatically active enzyme associated with the gene-specific chromatin complex.

In some embodiments, detecting the gene-specific chromatin regulatory protein complex comprises identifying one or more components of the gene-specific chromatin regulatory protein complex. In some embodiments, the enzyme associated with the gene-specific chromatin regulatory protein complex comprises a histone-modifying enzyme, a histone post-translational modification (PTM)-reading protein, a co-regulatory protein complex, and/or a transcriptional factor. In some aspects, the chemoprobe comprises an inhibitor of a histone-modifying enzyme or a histone PTM reader domain, optionally wherein the histone-modifying enzyme is selected from the group consisting of G9a and Ezh2, and optionally wherein the histone PTM reader domain is selected from the group consisting of a bromodomain (BRD) antagonist or acetyl-lysine (Kac). In some embodiments, the chemoprobe is selected from the group consisting of UNC0638, UNC1999 and I-BET, optionally wherein UNC0638 is immobilized on Sepharose beads (UNC2249), optionally wherein UNC0638 is biotinylated (UNC0965), optionally wherein UNC1999 is biotinylated (UNC2399), and/or optionally wherein I-BET is biotinylated (UNC3660A). In some embodiments, UNC0638 and/or UNC2249 and/or UNC0965 comprises a substrate-competitive inhibitor that selectively binds enzymatically active G9a, wherein UNC1999 and/or UNC2399 comprises a substrate-competitive inhibitor that selectively binds enzymatically active Ezh2, wherein UNC3660A comprises a substrate-competitive inhibitor that selectively binds enzymatically active BRD.

In some embodiments of the methods provided herein the enzymatically active enzyme associated with a gene-specific chromatin regulatory protein complex defines a transcriptional activity of a chromatin associated with a class of genes. In some embodiments, the isolated chromatin regulatory protein complex comprises a functional chromatin-modifying complex within chromatin associated with select genes. In some embodiments, the functional chromatin complex reveals how an activity-based protein complexome is assembled within the chromatin of defined transcriptional activity, and/or where it is localized in the genome.

In some embodiments the methods disclosed herein comprise contacting the sample with an affinity-tagged chemoprobe that selectively binds a chromatin modifier, a chromatin eraser, or a chromatin reader, optionally wherein the chromatin modifier is selected from G9a and Ezh2, and optionally wherein the chromatin reader is BRD. In some aspects, contacting the sample with two biotinylated chemoprobes, wherein a first chemoprobe selectively binds G9a, and wherein a second chemoprobe selectively binds a BRD, wherein corresponding protein complexes from transcriptional active genes or transcriptional repressive genes, respectively, can be isolated. In some aspects, the presence of a gene-specific chromatin regulatory protein complex in the sample is indicative of a disease phenotype. In some embodiments, the disease phenotype comprises a chronic inflammation-associated disease phenotype. In some embodiments, the presence of the gene-specific chromatin regulatory protein complex that is indicative of a disease phenotype comprises one or more biomarkers.

In some embodiments, the gene-specific chromatin regulatory protein complex is associated with disease-related genes selected from the group consisting of disease-causing, disease-suppressing, and tumor-suppressing genes.

In some embodiments, the chemoprobe is immobilized on a substrate. In some aspects, the substrate comprises a bead. In some aspects the methods further comprise immobilizing the chemoprobe in a pipette tip or multi-well plate. Still yet, in some aspects the chemoprobe is affinity tagged, optionally wherein the affinity tag comprises biotin.

In some embodiments the disclosed methods further comprise sequencing of one or more components of the gene-specific chromatin regulatory protein complex for biomarker identification.

In some embodiments, the sample is selected from the group consisting tissue, blood and plasma.

Still yet, in some embodiments, the disclosed methods further comprise identifying a gene-specific binding of a transcriptional factor. In some aspects, the disclosed methods further comprise identifying a co-regulator network.

Provided herein are also high-throughput methods for screening for a disease biomarker using chromatin activity-based chemoproteomics, comprising providing a chemoprobe, wherein the chemoprobe is substrate-competitive and selectively binds to an enzymatically active enzyme associated with a gene-specific chromatin regulatory protein complex, wherein the enzymatically active enzyme is present in a functional chromatin regulatory protein complex that is associated with a disease state, contacting one or more samples with the chemoprobe to screen for the presence of a functional chromatin regulatory protein complex comprising the enzymatically active enzyme in the one or more samples, whereby the functional chromatin regulatory protein complex is isolated from samples where it is present, and identifying one or more components of the isolated functional chromatin regulatory protein complex associated with the enzymatically active enzyme, whereby an identified component of the isolated functional chromatin regulatory protein complex associated with the enzymatically active enzyme comprises a biomarker for a disease state.

In some embodiments the methods further comprise defining an architecture of the isolated functional chromatin regulatory protein complex, wherein the defined architecture of the functional chromatin regulatory protein complex comprises a biomarker for disease state. In some embodiments the methods further comprise identifying a profile of interacting components within the isolated functional chromatin regulatory protein complex, wherein a defined profile of interacting components of the functional chromatin regulatory protein complex comprises a biomarker for disease state. Still yet, in some embodiments, the disclosed methods further comprise identifying a co-regulator network.

In some embodiments the biomarker is for a chronic inflammation-associated disease, optionally wherein the chronic inflammation-associated disease comprises a cancer.

In some embodiments, the contacting one or more samples with the chemoprobe further comprises contacting a first sample from a first subject and contacting a second sample from a second patient, optionally wherein the first subject is a healthy subject and the second subject has a disease phenotype.

In some embodiments, provided herein are components, architectures or profiles of components of a functional chromatin regulatory protein complex produced by the disclosed methods.

Accordingly, it is an object of the presently disclosed subject matter to provide chromatin-activity-based chemoproteomic (ChaC) methods and systems for disease marker development and/or discovery. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1 is a schematic illustration of the disclosed quantitative ChaC methodology for the characterization of the functional G9a interactome or complexome formed within the chromatin of differentially inflamed macrophages as an example in the present practice. The schematic illustrates the work-flow of the activity-based ChaC with an immobilized (bead) or epitope-tagged chemoprobe (inhibitor) exposed to nuclear extract from each set of differentially inflamed macrophages with the pull-down products subjected to LC-MS/MS.

FIGS. 2A-2C are blot images from immunoblot analysis of the G9a pulled down by UNC2249 from the differentially inflamed macrophages under different inflammatory conditions (N, NL, and TL). FIG. 2A is the input control. FIG. 2B is the δ-ACTIN is the loading control. FIG. 2C is the blot depicting results of the G9a pulled down by UNC2249 (upper bands indicate the long-isoform of G9a while the lower bands are the shorter forms).

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. DEFINITIONS

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Also encompassed are any and all nucleotide sequences that encode the disclosed amino acid sequences, including but not limited to those disclosed in the corresponding GENBANK® entries.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The terms “modulate” or “alter” are used interchangeably and refer to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the terms “modulate” and/or “alter” can mean “inhibit” or “suppress”, but the use of the words “modulate” and/or “alter” are not limited to this definition.

As used herein, the terms “inhibit”, “suppress”, “repress”, “downregulate”, “loss of function”, “block of function”, and grammatical variants thereof are used interchangeably and refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA. By way of example and not limitation, histone methyltransferases, such as G9a, can suppress transcription of a number of genes below that observed in the absence of histone methyltransferases.

The term “RNA” refers to a molecule comprising at least one ribonucleotide residue. By “ribonucleotide” is meant a nucleotide with a hydroxyl group at the 2′ position of a D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The term “transcription factor” generally refers to a protein that modulates gene expression, such as by interaction with the cis-regulatory element and/or cellular components for transcription, including RNA Polymerase, Transcription Associated Factors (TAFs), chromatin-remodeling proteins, reverse tet-responsive transcriptional activator, and any other relevant protein that impacts gene transcription.

The term “promoter” defines a region within a gene that is positioned 5′ to a coding region of a same gene and functions to direct transcription of the coding region. The promoter region includes a transcriptional start site and at least one cis-regulatory element. The term “promoter” also includes functional portions of a promoter region, wherein the functional portion is sufficient for gene transcription. To determine nucleotide sequences that are functional, the expression of a reporter gene is assayed when variably placed under the direction of a promoter region fragment.

“Chromatin” is a complex of macromolecules found in cells, comprising DNA, protein and RNA. Some of the primary functions of chromatin are 1) to package DNA into a smaller volume to fit in the cell, 2) to reinforce the DNA macromolecule to allow mitosis, 3) to prevent DNA damage, and 4) to control gene expression and DNA replication. The primary protein components of chromatin are histones that compact the DNA.

The term “chemoprobe” defines a small molecule that binds to the active site or substrate-binding site(s) of an enzyme, e.g., chromatin modifier, reader, or erasers. Therefore, only the functional-active form of a target enzyme will be selectively bound by a chemoprobe.

The terms “active”, “functional” and “physiological”, as used for example in “enzymatically active”, “functional chromatin” and “physiologically accurate”, and variations thereof, refer to the states of genes, regulatory components, chromatin, etc. that are reflective of the dynamic states of each as they exists naturally, or in vivo, in contrast to static or non-active states of each. Measurements, detections or screenings based on the active, functional and/or physiologically relevant states of biological indicators can be useful in elucidating a mechanism, or defining a disease state or phenotype, as it occurs naturally. This is in contrast to measurements taken based on static concentrations or quantities of a biological indicator that are not reflective of level of activity or function thereof.

As used herein, the terms “antibody” and “antibodies” refer to proteins comprising one or more polypeptides substantially encoded by immunoglobulin genes or fragments of immunoglobulin genes. The presently disclosed subject matter also includes functional equivalents of the antibodies of the presently disclosed subject matter. As used herein, the phrase “functional equivalent” as it refers to an antibody refers to a molecule that has binding characteristics that are comparable to those of a given antibody. In some embodiments, chimerized, humanized, and single chain antibodies, as well as fragments thereof, are considered functional equivalents of the corresponding antibodies upon which they are based. In some embodiments, the presently disclosed subject matter provides methods for identifying, characterizing and/or developing disease-related components of a gene-specific chromatin regulatory protein complex, wherein one or more antibodies can be used directly, or in assays related thereto, in the identification, characterization and/or isolation of such components.

The term “substantially identical”, as used herein to describe a degree of similarity between nucleotide sequences, peptide sequences and/or amino acid sequences refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide identity, when compared and aligned for maximum correspondence, as measured using a sequence comparison algorithm or by visual inspection. As used herein, the terms “detectable moiety”, “detectable label”, and “detectable agent” refer to any molecule that can be detected by any moiety that can be added to a chemoprobe, antigen, inhibitor, marker, reagent and/or antibody, or a fragment or derivative thereof, that allows for the detection of the chemoprobe, antigen, inhibitor, marker, reagent and/or antibody, fragment, or derivative in vitro and/or in vivo. Representative detectable moieties include, but are not limited to, chromophores, fluorescent moieties, radioacite labels, affinity probes, enzymes, antigens, groups with specific reactivity, chemiluminescent moieties, and electrochemically detectable moieties, etc. In some embodiments, the antibodies are biotinylated.

In some embodiments, a detectable moiety comprises a fluorophore. Any fluorophore can be employed with the compositions of the presently disclosed subject matter, provided that the conjugation of fluorophore results in a composition that is detectable either in vivo (e.g., after administration to a subject) and/or in vitro, and further does not negatively impact the ability of the chemoprobe, antigen, inhibitor, marker, reagent and/or antibody, or the fragment or derivative thereof, to bind to its epitope. Representative fluorophores include, but are not limited to 7-dimethylaminocoumarin-3-carboxylic acid, dansyl chloride, nitrobenzodiazolamine (NBD), dabsyl chloride, cinnamic acid, fluorescein carboxylic acid, Nile Blue, tetramethylcarboxyrhodamine, tetraethylsulfohodamine, 5-carboxy-X-rhodamine (5-ROX), and 6-carboxy-X-rhodamine (6-ROX). It is understood that these representative fluorophores are exemplary only, and additional fluorophores can also be employed. For example, there the ALEXA FLUOR® dye series includes at least 19 different dyes that are characterized by different emission spectra. These dyes include ALEXA FLUOR® 350, 405, 430, 488, 500, 514, 532, 546, 555, 568, 594, 610, 633, 635, 647, 660, 680, 700, and 750 (available from Invitrogen Corp., Carlsbad, Calif., United States of America), and the choice of which dye to employ can be made by the skilled artisan after consideration of the instant specification based on criteria including, but not limited to the chemical compositions of the specific ALEXA FLUOR®, whether multiple detectable moieties are to be employed and the emission spectra of each, the detection technique to be employed, etc.

In some embodiments, a detectable moiety comprises a cyanine dye. Non-limiting examples of cyanine dyes that can be conjugated to the chemoprobe, antigen, inhibitor, marker, reagent and/or antibody, fragments, and/or derivatives of the presently disclosed subject matter include the succinimide esters Cy5, Cy5.5, and Cy7, supplied by Amersham Biosciences (Piscataway, N.J., United States of America).

In some embodiments, a detectable moiety comprises a near infrared (NIR) dye. Non-limiting examples of near infrared dyes that can be conjugated to the chemoprobe, antigen, inhibitor, marker, reagent and/or antibody, fragments, and/or derivatives of the presently disclosed subject matter include NIR641, NIR664, NIT7000, and NIT782.

In some embodiments, biotinylated chemoprobes, antigens, inhibitors, markers, reagents and/or antibodies are detected using a secondary antibody that comprises an avidin or streptavidin group and is also conjugated to a fluorescent label including, but not limited to Cy3, Cy5, Cy7, and any of the ALEXA FLUOR® series of fluorescent labels available from INVITROGEN™ (Carlsbad, Calif., United States of America). In some embodiments, the chemoprobe, antigen, inhibitor, marker, reagent and/or antibody, fragment, or derivative thereof is directly labeled with a fluorescent label and enzymes, co-factors, peptides, molecules and/or cells that bind to the antibody are separated by fluorescence-activated cell sorting. Additional detection strategies are known to the skilled artisan.

For applications including but not limited to detection applications and imaging applications, the chemoprobes, antigens, inhibitors, markers, reagents and/or antibodies of the presently disclosed subject matter can be labeled with a detectable moiety. The detectable moiety can be any one that is capable of producing, either directly or indirectly, a detectable signal. For example, a detectable moiety can be a radioisotope, such as but not limited to ³H, ¹⁴C, ³²P, ³⁵S, ¹²⁵I, or ¹³¹I; a fluorescent or chemiluminescent compound such as but not limited to fluorescein isothiocyanate, rhodamine, or luciferin; or an enzyme, such as but not limited to alkaline phosphatase, β-galactosidase, or horseradish peroxidase.

### II. CHROMATIN-ACTIVITY-BASED CHEMOPROTEOMIC (CHAC) METHODS AND SYSTEMS FOR DISEASE MARKER DISCOVERY

Provided herein in some embodiments are methods for systemic identifications of multiple disease-related components of a gene-specific chromatin regulatory protein complex, which can in some embodiments provide for the development of one or more biomarkers that can be assessed for their use in diagnosis of a disease or phenotype. This methodology can in some aspects be referred to herein as chromatin activity-based chemoproteomics (ChaC). Such methods can in some embodiments utilize chemoprobes that are substrate-competitive and that selectively bind to an enzymatically active enzyme associated with a gene-specific chromatin regulatory protein complex. Because they target and isolate enzymatically active enzymes associated with gene-specific chromatin complexes, the disclosed ChaC approaches and methods can generate activity-based interactome data in a physiologically accurate manner and is generically applicable to dissect a variety of phenotypic chromatin architectures in the context of a functional protein complexome. Based on the information gathered using these approaches, new disease-related components and/or biomarkers for diseases and phenotypes can be identified and/or developed.

Activation of inflammation is the key host response to microbial challenge, yet excessive production of proinflammatory cytokines can lead to tissue/organ damage or autoimmune diseases (Coussens & Werb, 2002; Clevers, 2004). To minimize harmful effects caused by the continual presence of environmental stimuli, pre-exposed cells suppress cytokine production to become transiently unresponsive. However, this acquired immune tolerance is a double-edged sword, both protecting the host from infection/damage and, when dysregulated, contributing directly to various inflammation-associated pathologies (Kanterman et al., 2012). Development of tolerance to endotoxin or lipopolysaccharide (LPS) is a major molecular feature of the pathogenesis of many chronic diseases including asthma, sepsis, and cancer, as people experiencing endotoxin tolerance (ET) are immune-compromised (Biswas & Lopez-Collazo, 2009; Foster & Medzhitov, 2009). Recent studies have revealed that control of inflammation is achieved primarily epigenetically in a gene-specific manner, whereby, with prolonged LPS stimulation, chromatin associated with pro-inflammatory or ‘tolerizeable’ genes (T-genes) becomes transcriptionally silenced (Foster et al., 2007; Xie et al., 2013). However, prior to the instant disclosure, the functional constituents of the inflammation-phenotypic chromatin architecture that directly participate in transcription regulation of select genes were unknown.

Histone post-translational modifications (PTMs), such as acetylation, phosphorylation, methylation, determine the functional state of chromatin, which eventually control chromatin-mediated transcription (Johnson & Dent, 2013). Meanwhile, the levels of particular histone PTM(s) are regulated by corresponding histone-modifying enzymes, whose activities vary under different cell states (Badeaux & Shi, 2013). Thus, in some embodiments, methods are provided to screen for and identify disease-related components of chromatin regulator protein complexes, wherein in some embodiments the unique strength of chemoprobes is used to dissect various epigenetic protein machinery operating under defined phenotypic cell states.

By way of example and not limitation, and as a proof of concept, in some embodiments the disclosed ChaC methods and approaches are discussed in the context of ET. Immune cells develop ET after prolonged stimulation. ET increases the level of a repression mark H3K9me2 in the transcriptional-silent chromatin specifically associated with pro-inflammatory genes. However, prior to the instant disclosure it was not clear what proteins are functionally involved in this process. As discussed further herein, the disclosed ChaC approach provided for the dissection and identification of the functional chromatin protein complexes that regulate ET-associated inflammation. The disclosed ChaC methodology reveals how this repressome complex is built, localized, and regulated in the gene-specific manner. Of course, as set forth further herein, the disclosed ChaC approaches and methods are also applicable to dissect other functional protein complexes in the context of phenotypic chromatin architectures.

In some embodiments, the disclosed ChaC methods comprise the use of small molecules or chemoprobes to pull down or isolate and dissect the chromatin complexes localized at select classes of genes under defined chromatin states of different transcriptional activities. For example, in some embodiments, and as disclosed in further detail herein, a ChaC approach can use a small molecule such as UNC0638 that is a substrate-competitive inhibitor that selectively binds enzymatically active G9a. As a result, the disclosed ChaC methods, systems and compounds using UNC0638 immobilized on Sepharose beads (UNC2249) can in some embodiments reveal that the constitutively activated G9a coordinates an assembly of multiple, well-defined complexes within the chromatin associated with specific class(es) of genes, pro-inflammatory genes in particular, that are transcriptionally repressed under a defined inflammatory phenotype of endotoxin tolerance. This demonstrates that, based on the targeting nature of chemoprobes, the disclosed ChaC methods, systems and compounds can pull down chromatin complexes under defined chromatin states. For example, because defined histone PTMs such as H3K9me2 mark the transitional activity of the chromatin associated with particular class of the genes, which is determined or is correlated with the enzymatic activity of the corresponding modifier enzymes, select chemoprobes can specifically bind to the enzymatically active forms of these enzymes.

Because protein complexes are immunoprecipitated based on the abundance and not the activity of the bait protein, conventional antibody-dependent approaches for interactive screening are less sensitive in distinguishing in situ functional interactions, e.g., all G9a are pulled down together irrespective of different classes of genes with different chromatin states of transcriptional activities. Unlike most inhibitors that bind to the active site of their target enzymes, the disclosed ChaC chemoprobes can in some embodiments be a substrate-competitive ligand, so that the G9a-scaffolding can for example function to accommodate the functionally relevant protein interactions and ensure that they are preserved. Further, because of its high-specificity in binding to the active site of G9a (1050<15 nM), UNC0638/UNC2249, for example, can pull down G9a in proportion to its methylation activity and can homogeneously separate its activity-dependent protein complexes within chromatin with defined transcriptional activity from other non-enzymatically related protein interactions. Thus, in some embodiments no concern related to antibody specificity and cross-activity needs to be addressed with the disclosed methods and systems. For example, during ET, data (see Examples below) clearly showed that the methylation activity of G9a is differentially regulated within gene-specific chromatin under different inflammatory states with a higher population of enzymatically active G9a.

Thus, in some embodiments provided herein are method and/or systems for identifying or screening for one or more disease-related components of a gene-specific chromatin regulatory protein complex. In some embodiments, such methods can comprise providing a sample to be assayed, contacting the sample with an inhibitor, such as for example a chemoprobe, and detecting a gene-specific chromatin regulatory protein complex. Within the gene-specific chromatin regulatory protein complex that is detected, one or more disease-related components can then be identified. In some aspects, these disease-related components can be regarded as biomarkers for diseases or phenotypes.

That is, in some aspects the enzymatically active enzyme associated with a gene-specific chromatin regulatory protein complex defines a transcriptional activity of a chromatin associated with a class of genes. In some embodiments the isolated chromatin regulatory protein complex is a functional chromatin-modifying complex within chromatin associated with select genes. The functional chromatin complex can reveal how an activity-based protein complexome is assembled within the chromatin of defined transcriptional activity, and/or where it is localized in the genome.

In some aspects, the presence of a gene-specific chromatin regulatory protein complex in the sample is indicative of a disease phenotype. The disease phenotype can comprise a chronic inflammation-associated disease. Thus, further characterization of the gene-specific chromatin regulatory protein complex that is indicative of a disease phenotype can yield one or more biomarkers that can subsequently be assessed for use in disease diagnosis and/or screening. In some aspects, such gene-specific chromatin regulatory protein complexes can be associated with disease-related genes such as disease-causing, disease-suppressing, and/or tumor-suppressing genes. Therefore, in some embodiments, the disclosed ChaC methods and systems can yield biomarkers for these disease classes.

Correspondingly, the ChaC method can further comprise identifying and/or characterizing one or more components of the gene-specific chromatin regulatory protein complex. Such components, by association with a chromatin regulatory complex that is associated with a disease-related gene, can serve as biomarkers for such a disease or phenotype. Thus, in some aspects the disclosed methods can comprise chem-precipitation using a chemoprobe followed by sequencing, and in some embodiments deep-sequencing (deep-sequencing indicates that the total number of reads is many times larger than the length of the sequence under study), of the associated DNA elements, with such sequencing providing for the gene-specific localization of the protein complexes. The chemoprobe pull-down complexes can be characterized by the steps comprising SDS-polyacrylamide gel electrophoresis (SDS-PAGE), tryptic digestion, and/or on-beads digestion followed by mass spectrometry (e.g. liquid chromatography-mass spectrometry (LC-MS/MS)). Meanwhile, in some embodiments, either metabolic labeling using AACT/SILAC or chemical-labeling or label-free quantitation method can be used to provide ‘in-spectra’ quantitative markers to distinguish the phenotype-specific components of a chromatin regulatory complex.

In some embodiments, the chemoprobe, or inhibitor, is substrate-competitive and selectively binds to an enzymatically active enzyme associated with a gene-specific chromatin regulatory protein complex. As such, the gene-specific chromatin regulatory protein complex present in a sample is detected by virtue of an enzymatically active enzyme associated with the gene-specific chromatin complex. Thus, any chromatin complex that is not in an enzymatically active state will not be selectively detected or isolated by this method. Moreover, in some aspects, the enzymatic activity of an enzyme associated with a chromatin complex corresponds to the transcriptional activity of a gene controlled by the chromatin complex. Thus, an isolated functional chromatin complex can be based on the chemical nature of the small molecule or chemoprobe, wherein the complex can be isolated from genes with different transcriptional activities.

In some aspects, the enzyme, and particularly the enzymatically active form, screened for in the disclosed methods can comprise any enzyme involved in the regulation and/or modification of the chromatin complex, e.g. chromatin and/or histones. For example, in some embodiments a chemoprobe utilized in the disclosed methods can be selective against one or more of a histone-modifying enzyme (e.g. a methyltransferase), a histone post-translational modification (PTM)-reading protein, a co-regulatory protein complex, and/or a transcriptional factor. For example, a chemoprobe can comprise an inhibitor of a histone-modifying enzyme or a histone PTM reader domain. Such histone-modifying enzymes can be for example G9a and Ezh2. Such histone PTM reader domains can be a bromodomain (BRD) antagonist or acetyl-lysine (Kac).

Representative chemoprobe or chemoprobes used in the disclosed ChaC methods and systems can comprise UNC0638 (2-Cyclohexyl-N-(1-isopropylpiperidin-4-yl)-6-methoxy-7-(3-(pyrrolidin-1-yl)propoxy) quinazolin-4-amine; Vedadi et al., 2011). UNC0638 is a histone methyltransferase inhibitor (HMT), and can be used as a G9a/GLP selective methyltransferase chemical probe. In some embodiments and for purposes of facilitation the pull-down and/or isolation of chromatin complexes, UNC0638 can be immobilized on a substrate, such as for example a Sepharose bead. UNC0638 immobilized on a Sepharose bead can in some aspects be referred to as UNC2249. Moreover, in some aspects UNC0638 tagged or modified with a detectable moiety, such as for example biotin. In some embodiments, a biotinylated UNC0638 can be referred to as UNC0965. UNC0638, as well as its modified forms, UNC2249 and UNC0965, comprises a substrate-competitive inhibitor that selectively binds the enzymatically active form of G9a. In some embodiments, the chemoprobe or chemoprobes used in the disclosed ChaC methods and systems can comprise UNC1999 (CAS Number 1431612-23-5; 1-Isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide). UNC1999 is a substrate-competitive inhibitor that selectively binds the enzymatically active form of Ezh2, and in some embodiments Ezh1, both of which are lysine methyltransferases. In some aspects, UNC1999 can be biotinylated, in which case it can sometimes be referred to as UNC2399.

In some embodiments, the chemoprobe or chemoprobes used in the disclosed ChaC methods and systems can comprise I-BET, which in some embodiments can be biotinylated (UNC3660A). UNC3660A comprises a substrate-competitive inhibitor that selectively binds the Kac-recognizing BRD. In some embodiments, an affinity-tagged chemoprobe can be used that selectively binds a chromatin modifier, a chromatin eraser, or a chromatin reader. By way of example and not limitation a chromatin modifier can be G9a and/or Ezh2, while a chromatin reader can be BRD. In some aspects, two affinity-tagged, e.g. biotinylated, chemoprobes can be used in the disclosed ChaC methods. For example, a first chemoprobe can selectively bind G9a, and a second chemoprobe can selectively bind a BRD. By utilizing two chemoprobes protein complexes from transcriptional active genes and/or transcriptional repressive genes can be isolated.

In some embodiments, and for purposes of facilitating the pull-down and/or isolation of chromatin complexes, provided herein are chemoprobes that have been modified. For example, in some aspects a chemoprobe can be immobilized on a substrate. Such a substrate can comprise a bead, such as for example a Sepharose bead. Moreover, in some embodiments, a chemoprobe can be immobilized on or affixed to a pipette tip or multi-well plate. Such a configuration can facilitate high throughput assays and screening methods.

To elaborate, in some aspects, a method as disclosed herein can comprise immobilizing biotinylated chemoprobes in microtips (MSIA tips) for clinic assays. Such microtips can in some embodiments comprise immobilized streptavidin, Avidin, and/or NeutrAvidin (Thermo Mass Spec Immunoassay (MSIA) platform). Moreover, in some aspects, a chemoprobe can be immobilized in a multi-well plate, such as a 128- or 96-well plate, or other assay device or platform, for on-bead digestion of protein complexes for LC-MS/MS sequencing.

In some embodiments, a chemoprobe as disclosed herein, and as used in the methods disclosed herein, can further comprise a detectable moiety. Such a detectable moiety can facilitate detection and isolation of the chemoprobe and any chromatin or compound associated therewith. Thus, in some aspects the addition of a detectable moiety to a chemoprobe can be advantageous. For example, in some aspects a chemoprobe can comprise an affinity tag, which can for example be detected and/or isolated using an antibody or other highly-specific binding compound. Biotin is an example of an affinity tag. Biotinylation is the process of covalently attaching biotin to a protein, nucleic acid or other molecule, such as for example a chemoprobe. Biotin binds to streptavidin and avidin with an extremely high affinity, fast on-rate, and high specificity, which can then facilitate the isolation of the biotinylated molecules. Other detectable moieties can comprise chromophores, fluorescent moieties, radioacite labels, affinity probes, enzymes, antigens, groups with specific reactivity, chemiluminescent moieties, and electrochemically detectable moieties.

In some aspects, a sample to be screened or tested can be selected from a subject or patient, such as a mammal or human. Such samples can comprise any biological sample for which testing is desired, including for example tissue (e.g. by biopsy), blood and/or plasma. Samples can be processed as discussed hereinbelow in the Examples, and can in some embodiments comprise the preparation of nuclear extracts.

As noted above, the disclosed methods can comprise the identification and/or characterization of disease-related components of a chromatin regulatory protein complex, which can serve as a biomarker(s) once associated with a given disease, phenotype or condition. In addition to the identification of individual chromatin regulatory complex components which can serve as biomarkers, the disclosed methods can also provide for the identification of transcriptional factors that are present or associated with chromatin complexes in particular disease states. Moreover, a co-regulator or co-regulatory network can be identified, such that the presence of the network can serve as a biomarker. Additionally, profiles of complex components, or associations of particular components, can serve as a biomarker. Moreover, the disclosed methods can provide for the defining of an architecture of an isolated functional chromatin regulatory protein complex. A defined architecture of a functional chromatin regulatory protein complex can then serve a biomarker for disease state or phenotype. Defining the architecture of a functional chromatin regulatory protein complex can in some embodiments comprise characterizing the components that make up the chromatin complex and their relationships to one another, wherein the components can comprise transcriptional factors, histone-modifying enzymes, histone PTMs, PTM readers, and chromatin regulators.

In some embodiments, high-throughput methods for screening for one or more disease biomarkers using chromatin activity-based chemoproteomics are provided. Such high-throughput methods can comprise providing a chemoprobe, wherein the chemoprobe is substrate-competitive and selectively binds to an enzymatically active enzyme associated with a gene-specific chromatin regulatory protein complex, wherein the enzymatically active enzyme is present in a functional chromatin regulatory protein complex that is associated with a disease state. Such chemoprobes can be similar to those described herein with regard to other screening and/or identification methods.

The high-throughput methods can further comprise contacting one or more samples with the chemoprobe to screen for the presence of a functional chromatin regulatory protein complex comprising the enzymatically active enzyme in the one or more samples, whereby the functional chromatin regulatory protein complex is isolated from samples where it is present. One or more components of the isolated functional chromatin regulatory protein complex associated with the enzymatically active enzyme can be identified, whereby an identified component of the isolated functional chromatin regulatory protein complex associated with the enzymatically active enzyme comprises a biomarker for a disease state.

In some embodiments, the high-throughput screening method can comprise contacting a first sample from a first subject and contacting a second sample from a second patient, wherein the first subject is a healthy or control subject and the second subject has a disease phenotype or a phenotype of interest. In such a configuration, the high-throughput screening method can provide for the comparison between a normal or healthy patient and a sick or diseased patient (or at least a patient believed to be suffering from a disease). In such an embodiment the comparison of chromatin regulatory complexes between the two patient phenotypes can facilitate the identification and/or characterization of biomarkers for the phenotype or disease. Indeed, in some aspects, particularly a high-throughput arrangement, a plurality of patient samples (some from healthy patients and some from sick patients) can be screened so as to provide sufficient data to identify and/or characterize one or more biomarkers that can be assessed for their association with a disease or phenotype.

The ChaC approach disclosed herein can generate activity-based interactome data in a physiologically accurate manner and is generically applicable to dissect a variety of phenotypic chromatin architectures in the context of a functional protein complexome. In some aspects, this ChaC methodology that can reveal the inflammation-phenotypic, functional constituents of the chromatin writer complexome that regulates gene-specific transcription. Thus, the functional constituents of the inflammation-phenotypic chromatin architecture that directly participate in transcription regulation of select genes in inflammation-associated diseases can be identified, characterized and profiled to generate biomarkers. Such biomarkers can be assessed for their potential use in diagnosing a phenotype or disease.

In some embodiments provided herein are biomarker articles, e.g. compounds or compositions, for a chronic inflammation associated disease. Such a biomarker article can comprise a chemoprobe, wherein the chemoprobe selectively binds to an enzymatically active enzyme associated with a chromatin regulatory protein complex, wherein the enzymatically active form of the enzyme is correlated with a chronic inflammation associated disease. Such a biomarker article can further comprise a detectable moiety associated with the chemoprobe, and in some embodiments a substrate to which the chemoprobe is affixed. In some embodiments, a panel or series of chemoprobes or identified biomarkers can be provided.

As discussed above in the context of the methods, chemoprobes for the biomarker articles can comprise an inhibitor of a methyltransferase, such as for example G9a and Ezh2. In some aspects, the chemoprobe can selectively bind a chromatin modifier, eraser, or reader.

In some aspects, a chemoprobe used in a biomarker article can comprise a detectable moiety, such as for example a radioactive label, an affinity probe, a luminescent probe, a fluorescent probe, or contrast agent.

### III. SUBJECTS

The subject screened, tested, or from which a sample is taken, is desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which screening is desirable, particularly agricultural and domestic mammalian species.

The disclosed methods are particularly useful in the testing, screening and/or treatment of warm-blooded vertebrates. Thus, the presently disclosed subject matter concerns mammals and birds.

More particularly, provided herein is the testing, screening and/or treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, provided herein is the treatment of livestock, including, but not limited to, domesticated swine (pigs and hogs), ruminants, horses, poultry, and the like.

In some embodiments, the subject to be used in accordance with the presently disclosed subject matter is a subject in need of treatment and/or diagnosis. In some embodiments, a subject can have or be believed to an inflammation-associated disease, condition or phenotype.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-10

Reagents and Mice.

Lipopolysaccharide (LPS; Escherichia coli 0111:B4) was purchased from InvivoGen (InvivoGen, San Diego, Calif., United States of America). An HEK 293 stable TLR4-MD2-CD14 cell line was also purchased from InvivoGen. All components of cell culture media and protease inhibitor cocktails were purchased from Sigma (St. Louis, Mo., United States of America). Fetal bovine serum was obtained from GIBCO® (Life Technologies, Inc., Grand Island, N.Y., United States of America). Trypsin was purchased from Promega (Madison, Wis., United States of America). All chemicals were either sequence or HPLC grade unless specifically indicated. Antibodies against G9a cat#07-551 (1:1,000), H3K9me2 #07-441 (1:1,000), H3K4me3 #07-473 (1:1,000) and H3K4me2K9me2 #07-1843 (1:2,000) were purchased from Millipore (EMD Millipore, Billerica, Mass., United States of America). Antibodies for c-Myc (N-262) #sc-764x (1:2,000), Brg1 (H-88) #sc-10768x (1:2,000), HDAC1 #sc-7872 (1:1,000), MTA1 #sc-373765 (1:1,000), YY1 (H-414) #sc-1703 (1:1,000), SIRT-1 #sc-15404 (1:1,000) and b-Actin #sc-1616 (1:2,000) were from Santa Cruz Biotechnology, Inc. (Dallas, Tex., United States of America). Antibodies for H3 #ab1791 (1:5,000), and g-Tubulin #ab11316 (1:5,000) were from Abcam, PLC (Cambridge, United Kingdom). Antibodies for H3K36me2 #39255 (1:5,000) and H3K27me3 #39156 (1:5,000) were purchased from Active Motif (Carlsbad, Calif., United States of America). Antibodies for H3K27me2 #PA5-17376 (1:2,000) were purchased from Pierce Antibody Products (Thermo Fisher Scientific, Inc., Rockford, Ill., United States of America). Antibodies for p-p65 (S536) #3033 (1:1,000) were from Cell Signaling Technology, Inc. (Beverly, Mass., United States of America). Antibodies for NSD3 #GTX109396 (1:1,000) were from GeneTex (GeneTex, Inc., Irvine, Calif., United States of America). Antibodies for CtBP2 #612044 (1:2,000) were from BD Biosciences (East Rutherford, N.J., United States of America). Anti-HA (clone HA-7) (1:4,000) and anti-flag M2 (clone M2) (1:4,000) antibodies were from Sigma. Male or female mice (6 to 8 weeks old; C57BL/6 strain) were used. All mouse procedures in this study were approved by the Institutional Animal Care & Use Committee (IACUC) at the University of North Carolina at Chapel Hill, N.C., United States of America.

BMDM Culturing and AACT/SILAC Labelling.

Bone marrow progenitors were rinsed out from femur and tibia bones of C57BL/6 wild-type mice. Red blood cells were removed by ACK Lysing Buffer (Gibco). Bone marrow cells were cultured on petri dishes for 6 days in the DMEM supplemented with 10% fetal bovine serum, 20% L929-conditioned media, either regular or AACT-containing media. On day 7, BMDMs were lifted with cold TEN buffer (40 mM Tris-HCl pH 7.4, 1 mM EDTA, 150 mM NaCl) and replated on tissue-culture treated plates.

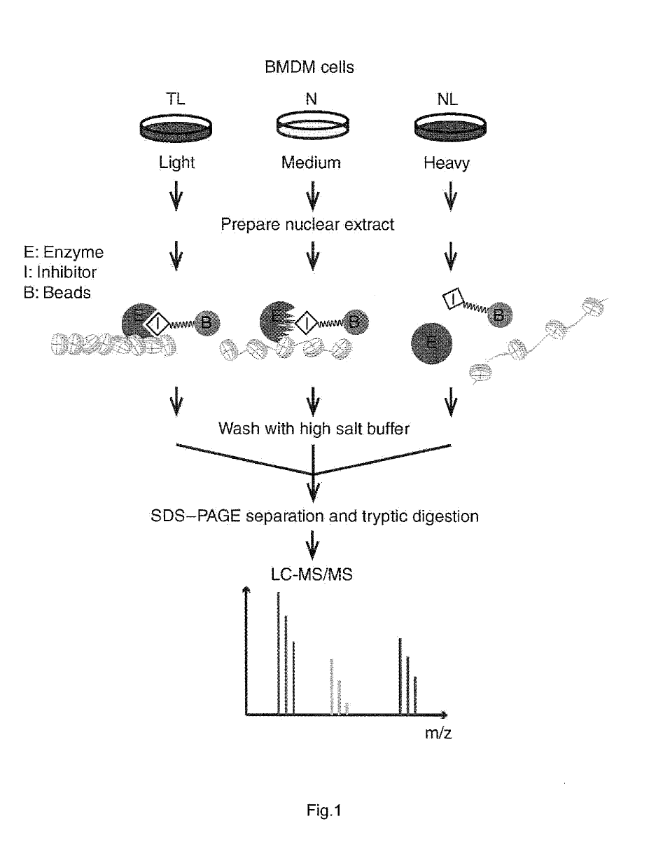

Stable isotope labelling growth media were prepared (Xie et al., 2013). The growth media in depletion of lysine and arginine were supplemented with either 13C6-lysine (K6)/13C6-arginine (R6) or 13C6 15N2-arginine (K8)/13C6 15N4-arginine (R10), respectively (Sigma-Aldrich cat#643459, cat#643440, cat#608041, cat#608033). Primary BMDMs or Raw264.7 cells were either left unstimulated (native; N; FIG. 1), or subjected to a single LPS stimulation at 1 mgml⁻¹, or first primed with 100 ng ml⁻¹ LPS to induce ET for 24 h (FIG. 1), followed by the second LPS challenge at 1 mgml⁻¹ (TL; FIG. 1). Cells were grown at −37° C. in a humidified atmosphere and 5% carbon dioxide in air.

Histone Sample Preparation.

Core histone proteins were prepared by Histone Purification Mini Kit (Active Motif; cat#40026). Adhered cells were washed with prewarmed serum-free media twice and were then harvested. The pellet was lysed with 1.5 ml extraction buffer per 2×10⁷ cells and homogenized by repetitive pipetting. The cell lysate was processed under agitation for 2 h at 4° C. before the crude histone was clarified, neutralized and loaded onto the column. Histone proteins were purified by a C8 column and were precipitated by 4% perchloric acid according to the manufacturer's instructions and were resolved in HPLC water. All steps were processed on ice or in cold room, for the maximum preservation of histone modifications.

HPLC Purification of Histone and Top-Down Mass Spectrometry.

Histone proteins were injected onto a C-8 column with a particle size of 7 mm and the inner diameter of 2.1 mm (Perkin Elmer, Inc., Waltham, Mass., United States of America; cat#07110060) using an Agilent 1100 series RPHPLC (Agilent, Santa Clara, Calif., United States of America). The core histone proteins were separated using the gradient: 35% to abut 60% acetonitrile/0.1% trifluoroacetic acid for 75 minutes. Protein elution was monitored by ultraviolet absorption at a wavelength of 214 nm. Histone fractions were collected, lyophilized and then subjected to mESI-FTICR-MS analysis (Gardner et al., 2011).

The use of a LTQ Orbitrap™ Velos™ mass spectrometer (Thermo Scientific) was also investigated to perform top-down MS analysis of inflammationphenotypic HPLC-purified, full-length H3.1. Mass analysis was achieved in both Full-MS, SIM of +19 charge state and electron transfer dissociation-tandem mass spectrometry modes (Syka et al., 2004) with an instrument resolution setting of 120,000 at m/z 400 Da. All samples were dissolved in a solution of 50:50 methanol:water and 1% formic acid and introduced to the mass spectrometer via direct infusion at a flow of 8 ml/min⁻¹. ETD was performed specifically on the isotopic clusters of two peaks (average m/z of 809.29 Th and m/z of 810. 03 Th) for the +19 charge state that correspond to various isoforms showing abundance variations, respectively, under N, NL and TL states. The lower m/z range: 200-780 Th product ions were processed manually in all electron transfer dissociation-tandem mass spectrometry spectra by assigning sequence ions to theoretical masses corresponding to a diverse set of combinatorial PTMs (Tian et al., 2012) that includes R2(me2), R2(me), K4(ac), K4(me2), K4(me), K9(ac), K9(me2), K9(me), K9(me3), K14(ac), K14(me), K14(me2), K27(ac), K27(me), K27(me2), K36(ac), K36(me) and K36(me2).

Nuclear Extraction.

Nuclear extract was prepared (Wysocka, 2006). BMDM cells were collected and resuspended in hypotonic buffer A (10 mM HEPES pH 7.9, 1.5 mM MgCl₂, 10 mM KCl, 1 mM phenylmethylsulfonyl fluoride (PMSF), 1 mM DTT and protease inhibitor cocktail). After incubating for 10 min on ice, the swollen cell pellet was spun down and resuspended in two volumes of Buffer A and homogenized to release cytosolic fraction. Nuclei were collected by centrifugation at 14,000 r.p.m. for 10 min and homogenized with 10 strokes in Buffer C (20 mM HEPES pH 7.9, 1.5 mM MgCl₂, 0.2 mM EDTA, 420 mM NaCl, 25% Glycerol, 1 mM PMSF, 1 mM DTT and protease inhibitor cocktail). Extraction was allowed to proceed under agitation for 30 min at 4° C. before the nuclear extract was clarified by centrifugation at 14,000 r.p.m. for 30 min at 4° C. The extract was dialyzed overnight to 50 volumes buffer D (20 mM HEPES pH 7.9, 0.2 mM EDTA, 100 mM KCl, 20% Glycerol, 1 mM PMSF, 1 mM DTT and protease inhibitor cocktail). Adding Triton-X-100 to a final concentration of 0.1% and centrifuging again, the supernatant was the nuclear extract.

Chromatin Activity-Based Chemoproteomics

An aminopropyl linker was added for coupling UNC0638 (Vedadi et al., 2011) to ECH sepharose 4B beads (GE Healthcare Bio-Sciences, Pittsburgh, Pa., United States of America; cat#17-0571-01). UNC0638 immobilized on sepharose beads were washed in Buffer D with 0.1% Triton-X-100 twice before addition into the nuclear extract. After incubation overnight, the beads were washed with 250 mM KCl buffer (20 mM HEPES pH 7.9, 0.2 mM EDTA, 250 mM KCl, 20% Glycerol, 1 mM PMSF, 1 mM DTT and protease inhibitor cocktail) five times. The pull-down products were eluted by boiling the beads in 2×SDS-polyacrylamide gel electrophoresis loading buffer for 10 min. For histone peptide pull-down experiments, histone H3 N-tail peptides was synthesized with a biotin tag, then the peptide was conjugated to streptavidin beads and the pull-down experiment was carried out in a similar way as UNC0638 pull-down except for that the washing buffer was substituted by a low-stringent buffer containing 100 mM KCl. The pull-down products were resolved by SDS-polyacrylamide gel electrophoresis, either for immunoblotting or followed by in-gel tryptic digestion before MS analysis.

Stable shRNA-Mediated Knockdown of G9a

The lentiviral plasmids for the shRNA targeting mouse Ehmt2 (G9a) in a vector pLKO.1 were purchased from Thermo Scientific Open Biosystems (Thermo Scientific). A pLKO.1 EV without shRNA sequence was used as the wild-type control. To generate virus, pLKO.1-shRNA plasmids were cotransfected into 293T cells with ViraPower™ Mix (Invitrogen) by jetPRIME in vitro DNA and short interfering RNA transfection reagent (Polyplus-transfection SA, Illkirch, France). Viral supernatants were collected 24 and 48 h following transfection and were used to transduce Raw264.7 cells by spinoculation. Forty-eight hours after the transfection, 8 mg/ml⁻¹ puromycin was added to initially select puromycin-resistant clones. The stable clones were maintained in the media containing 4 mg/ml⁻¹ puromycin. The level of G9a expression was examined for individual clones. Those clones with at least 90% knockdown efficiency were used in the experiments.

Quantitative Proteomic Profiling Protein Expression During ET

The pair of Raw264.7 cell lines either stably expressing shRNA for G9a KD, or the wild-type with EV were cultured for AACT labelling as described above. Both sets of the cells were primed with a low-dose LPS at 100 ng/ml⁻¹ for 24 h and then challenged with a high-dose LPS at 1 mg/ml⁻¹ to induce ET. Cells were harvested 2 h after LPS challenge and lysed in buffer containing 8M urea, 50 mM Tris-HCl pH 8.0, 75 mM NaCl, 1 mM MgCl₂, 500U Benzonase and protease inhibitor cocktail. Equal amounts (0.5 mg) of the lysate from each pool were mixed. The mixed protein pool was first reduced with DTT followed by cysteine alkylation with iodoacetamide, and then diluted four-fold by 25 mM Tris-HCl pH 8.0, 1 mM CaCl₂. The diluted protein mixture was digested with trypsin (Promega #V5113) at 37° C. overnight and then replenished trypsin to further digestion for another 6 h. The peptides were desalted on SEP-PAK® Light C18 cartridge (Waters Corporation, Milford, Mass., United States of America) and dried. The peptides were dissolved in 10 mM ammonium formate pH 10.0 and subjected to high-pH RPLC separation performed with an Xbridge C18, 250×4.6 mm column containing 5 mM particles and equipped with a 20×4.6 mm guard column (Waters). The mobile-phase A consisted of 10 mM ammonium formate (pH 10.0) and B 10 mM ammonium formate (pH 10.0) with 90% acetonitrile. Peptide separation was accomplished using the following gradient: from 0 to 5% B in 5 min, from 5 to 35% B in 30 min, from 35 to 70% B in 7.5 min and held at 70% B for an additional 5 min. Sixty fractions were collected and concentrated into 30 fractions by combining fractions 1, 21, 41; 2, 22, 42. The samples were dried and desalted on StageTip containing 4×1 mm C18 extraction disk (3M Company, St. Paul, Minn., United States of America).

Mass Spectrometry Analysis of ChaC Pull-Down Products

Peptide samples were dissolved in 0.1% formic acid and subjected to nanoLC-MS/MS analysis by using an online nanoLC ultra2D system (Eksigent, Redwood City, Calif., United States of America) coupled to an LTQ Orbitrap Velos mass spectrometer (Thermo Scientific) (Xie et al., 2013). LC-MS experiments were performed in a data-dependent mode with Full-MS (externally calibrated to a mass accuracy of <5 p.p.m. and a resolution of 60,000 at m/z 400) followed by CID MS/MS of the top 10 most intense ions.

Mass spectra processing and peptide identification were performed on the Andromeda search engine in MaxQuant™ software (Ver. 1.2.0.18) (Cox et al., 2011) against a mouse Uniprot™ database. All searches were carried out with cysteine carbamidomethylation as a fixed modification, while methionine oxidation and protein amino-terminal acetylation as dynamic modifications. Peptides were confidently identified using a target-decoy approach with a peptide false discovery rate (FDR) of 1% and a protein FDR of 1%. Data processing and statistical analysis were performed on Perseus (Version 1.2.0.17) (Cox et al., 2011). AACT/SILAC quantitation (3-plex KORO, K6R6, K8R10 or 2-plex KORO, K8R10) analysis was carried out similarly as previous described (Xie et al., 2013). The AACT ratios of the proteins were derived from the comparison of the extracted ion chromatogram peak areas of all matched light (L) peptides with those of the medium (M) or heavy (H) peptides. The ratios of M/L or H/L were also verified by visual inspection of the raw mass spectra.

Biological processes are categorized by DAVID Bioinformatics Resources 6.7 (National Institute of Allergy and Infectious Diseases (NIAID), NIH). Protein to protein interaction network analysis was performed by an online bioinformatics tool STRING (Search Tool for the Retrieval of Interacting Genes/Proteins). TF regulatory network was preceded by IPA (Ingenuity pathway analysis).

RNA Preparation and Quantitative PCR.

Total RNA was isolated using RNeasy kit (Qiagen, N.V., Venlo, Limburg). First-strand cDNA was synthesized by M-MLV reverse transcriptase (Promega, car#M170B) and diluted 10 times for quantitative PCR. Real-time PCR was performed using SYBR Green Master Mix (Thermo Fisher, cat#0221). All measurements were normalized to GAPDH and represented as relative ratios.

Sample Collection of the Macrophage Secretome.

Primary BMDM cells were primed with 250 nM UNC0638 or DMSO for 16 h. Referring now to FIG. 1, cells were then either left unstimulated (N) or subjected to a single LPS challenge at 500 ng/ml⁻¹ for indicated hours (NL) or first primed with 100 ng/ml⁻¹ LPS to induce the ET for 24 h, followed by the second LPS challenge at 500 ng/ml⁻¹ for indicated hours (TL). Before the LPS challenge, cells were washed once and cultured in serum-free DMEM without phenolred, containing 1 mM sodium pyruvate and 10 mM L-glutamine. The secreted proteins were harvested and prepared (Meissner et al., 2013).

Label-Free Quantitative Analysis of Macrophage Secretome.

LFQ secretome analyses were performed via reversed phase LC-MS/MS using a Proxeon 1000 nano LC system coupled to an Q Exactive mass spectrometer (Thermo Scientific). The Proxeon system was configured to trap peptides using a 3-cm long 100 mm i.d. C18 column at 5 ml/min⁻¹ liquid flow that was diverted from the analytical column via a vent valve, while elution was performed by switching the valve to make the trap column in line with a 15 cm long, 75 mm i.d., 3.5 mm, 300-A particle; C18 analytical column. Analytical separation of all the tryptic peptides was achieved with a linear gradient of 2% to 35% buffer B over either 240 min at a 300 nl/min⁻¹ flow rate, where buffer A is aqueous solution of 0.1% formic acid and buffer B is a solution of acetonitrile in 0.1% formic acid.

LC-MS experiments were also performed in a data-dependent mode with Full-MS (externally calibrated to a mass accuracy of <5 p.p.m. and a resolution of 70,000 at m/z 200) followed by HCD-MS/MS of the top 20 most intense ions. High-energy collision-activated dissociation (HCD)-MS/MS was used to dissociate peptides at normalized collision energy of 27 eV in the presence of nitrogen bath gas atoms. All secretome samples comprising three biological replicates were subjected to single-shot independent LC-MS runs resulting in the production of 48 LC-MS runs for global peptide analysis. Mass spectra were processed and peptide identification was performed using Andromeda search engine found in MaxQuant™ software ver. 2.2.1. (Max Planck Institute, Germany). All protein database searches were performed against the uniprot mouse protein sequence database. Peptides were identified with a target-decoy approach using a combined database consisting of reverse protein sequences uniprot mouse sequence and common repository of adventitious proteins (cRAP). The cRAP database was obtained from the Global Proteome Machine available online. Peptide identification was made with a FDR of 1% while peptides were assigned to proteins with a protein FDR of 5%. A precursor ion mass tolerance of 20 p.p.m. was used for the first search that allowed for m/z retention time recalibration of precursor ions that were then subjected to a main search using a precursor ion mass tolerance of 5 p.p.m. and a product ion mass tolerance 0.5 Da. Search parameters included up to two missed cleavages at KR on the sequence and oxidation of methionine as a dynamic modification. All protein and peptide identifications are reported by filtering for reverse and contaminant proteins.

Label-free quantitation was based on the peak area (Cox et al., 2008; Gunawardena et al., 2013; Gunawardena et al., 2011; Porro et al., 2007). The measured area under the curve of m/z and retention time aligned extracted ion chromatogram (XIC) of a peptide was performed via the label-free quantitation module found in MaxQuant™ (vera 1.3.0.5) (Cox et al., 2008). All replicates (biological and technical replicates) of each experimental conditions: N, NL-16 h, TL-16 h, were included in the LFQ experimental design with protein-level quantitation and normalization performed using unique and razor peptide features corresponding to identifications filtered with a peptide FDR of 0.01 and protein FDR of 0.05. The MaxQuant™ protein groups and evidence files were processed using the statistical analysis and visualization features found in Perseus™ (ver. 1.4.1.3) (Max Planck Institute, Germany).

Tagged Immunoprecipitation

HEK 293 stable TLR4-MD2-CD14 cell line (Invivogen) was transfected with indicated constructs by jetPRIME™ transfection reagent (Polyplus, cat#114-15). The cells were harvested from indicated conditions and lysed with RIPA buffer containing 20 mM Tris-HCl, 137 mM NaCl, 2 mM EDTA, 10% glycerol, 0.5% NP-40 and 1 mM PMSF. Cell lysates were incubated with affinity beads for overnight at 4° C. on rotator. Affinity beads were washed with RIPA buffer four times before subjected to immunoblot assay.

Chromatin Immunoprecipitation.

ChIP experiments for studying histone modifiers and TFs were carried out in dual crosslinking way (Porro et al., 2007) with minor modifications. Cells were fixed using 2 mM DSG (disuccinimidyl glutarate, ProteomChem #c1104) for 45 min. Formaldehyde (1% v/v) was then added and incubated for 15 min. Crosslinking reactions were quenched by adding glycine to 0.1 M, followed by PBS washing. Cells were scraped and nuclei were pelleted by 0.5% NP-40 lysis buffer (50 mM pH 8.0 Tris-HCl, 85 mM KCl, 0.5% NP-40). The nuclear fraction was extracted with RIPA buffer (50 mM pH 8.0 Tris-HCl, 150 mM NaCl, 1 mM EDTA, 1% Triton-X-100, 0.1% SDS, 0.1% sodium deoxycholate) and sonicated to 200 to 1,000 bp DNA fragments, then incubated overnight with the antibody. The antibody complexes were linked to protein A/salmon sperm DNA beads (Cell Signaling, cat #9007) and washed thoroughly. The complexes were then eluted from the beads by 1% SDS, and incubated with NaCl to reverse the crosslinked products. Later, proteins were digested by protease-K and the released DNA was purified by QIAquick PCR purification Kit (Qiagen, cat #28104), and processed to quantitative PCR assay. ChIP experiments for studying histone post-PTMs were started from a one-step fixation of 1% (v/v) formaldehyde for 10 min, followed by the same steps as dual crosslinking ChIP described above.

ChIP-seq.

ChIP experiments against c-Myc antibody (Santa Cruz cat#sc-764x) were processed in G9a KD stable cell line and wild-type control Raw264.7 cells, respectively, following the one-step formaldehyde fixation procedure. ChIP's DNA from two cell lines and INPUT from wild-type cells were subjected to library construction using ThruPLEX-FD Prep Kit (Rubicon Genomics, Ann Arbor, Mich., United States of America; cat#R40012-08). DNA libraries are cleaned up with Agencourt AMPure XP beads (Beckman Coulter, Inc., Indianapolis, Ind., United States of America; cat#A63880), quantified by Qubit 2.0 fluoremeter and qualified by Experion automated electrophoresis system (BIO-RAD, Inc., Hercules, Calif., United States of America). Three libraries were pooled and subjected to deep sequencing on Illumina Hiseq 2000. ChIP-seq data were aligned using Bowtie 2 (Version 2.1.0) (Langmead et al., 2012) to build version NCBI37/mm9 of mouse genome. ChIP-seq-enriched region over INPUT background were identified with peaking finding algorithm of MACS70 (Model based analysis of ChIP-seq).

G9a Activity Assay

293/TLR4-MD2-CD14 cells were transfected with tagged G9a plasmids and tagged-c-Myc plasmids as indicated. The next day, the transfected cells were primed with 100 ng/ml⁻¹ LPS for 24 h and then challenged with 1 mg/ml⁻¹ LPS for another 45 min. Cells were harvested and subjected to tagged IP with anti-flag affinity beads. After overnight incubation, anti-flag beads were washed with reaction buffer (Tris-HCl 20 mM pH 8.0, 25 mM NaCl, 0.025% Tween-20 and 1 mM DTT). In vitro assay was carried out as previous described (Vedadi et al., 2011). In brief, anti-flag beads were incubated with 5 mM S-adenosyl methionine and 2 nM fluorescent labelled H3 N-tail peptides in reaction buffer. After incubation for 2 h at 25° C., 40 pg/ml⁻¹ endoproteinase-LysC (Endo-LysC) was added and allowed to digest the remaining unmethylated substrate peptides for 1 h before separation on the Caliper LabChIP EZ Reader II (Caliper Life Sciences, Hopkinton, Mass., United States of America). The histone H3 lysine 9 methyltransferase activity was represented as the percentile of peptide substrate being methylated. Recombinant G9a was applied as the positive control. The reactions lacking catalytic enzyme or methyl donor S-adenosyl methionine were applied as negative controls.

NF-κB Reporter Assay.

293/TLR4-MD2-CD14 cells were seeded on 24-well plates 1 day before transfection so that they will be 90% confluent at the time of transfection. The next day, the cells were transfected with 225 ng pGL2-ELAM promoter Firefly luciferase transgene plasmid and 450 ng plasmids indicated. The pRL-TK plasmid, which expresses Renilla luciferase, was transfected into cells at 75 ng as an internal control to normalize transfection efficiency and sample handling. The cells were stimulated with 1 mg/ml⁻¹ LPS for 6 h before harvesting. The activities of the two kinds of luciferase were measured with their respective substrates with a dual luciferase assay kit (Promega). The luminescence reads reflecting NF-kB activity were obtained from the division of Firefly luciferase reads by Renilla luciferase reads. The data represented in the corresponding figures were the luminescence normalized to the control sample transfected with EV instead of functional plasmids. y axis indicates relative NF-kB activity. The error bar represents the s.d. of triplicates. Similar results were obtained in three independent biological experiments.

Measurements of c-Myc Stability and Degradation.

Raw264.7 or TLR4/MD2/CD14 293 cell lines were primed with 100 ng/ml⁻¹ LPS for 24 h and then treated with 1 mg/ml⁻¹ cycloheximide (Sigma-Aldrich; #C4859) for indicated time. Cells were harvested and subjected to immunoblot analysis by using anti-c-Myc antibody. Exposed films of immunoblot were scanned and quantified by ImageJ densitometry software. The intensity of each band was plotted in scatter plot and c-Myc half-life was determined on computed curve. Raw264.7 cells were primed with 100 ng/ml⁻¹ LPS for 24 h and then treated with 10 mM MG-132 for time indicated. C-Myc accumulation was reflected in immunoblot against c-Myc by short- and long-exposure. b-Actin and g-Tubulin were represented as loading controls.

Clonogenic Survival Assay

Raw264.7 cells were counted and plated in six-well cell culture plates with 50, 100 and 200 viable cells in duplicated wells. Cells were primed with 100 ng/ml⁻¹ LPS for 48 h to reach the condition of ET and replenished with fresh media. UNC0638 (1 mM) was replenished into the media. After 9-day cell growth, cell colonies were washed with PBS, fixed with a mixture of methanol-acetic acid (3:1 in volume ratio) for 10 min, stained with 10% Giemsa (Ricca Chemical Company, Arlington, Tex., United States of America) for 30 min, gently washed with water and air dried. The colonies containing more than 20 cells were counted and the percentage of the survived cells under each condition was determined compared with the number of colonies on non-LPS or UNC0638-treated plates.

### Example 1

### Histone H3 PTM is Reprogrammed in LPS-Tolerant Macrophages

To determine how histone H3 is differentially modified during the macrophage response to acute versus chronic LPS stimulation, top-down MS (Zhao et al., 2010) was first used to map the PTM landscape of full-length H3 from bone marrow-derived macrophages (BMDMs) under the following inflammatory states (Xie et al., 2013): non-stimulated (native) cells, cells stimulated with a single high-dose LPS (LPS-responsive), cells primed with a low-dose LPS (LPS-tolerant), and LPS-tolerant cells re-stimulated with a high-dose LPS that mimics ET. Following histone extraction and HPLC purification of individual core histones, different combinations of PTMs on full-length H3.1 were identified by top-down FTICR-MS based on the accurate mass measurements (Zhao, et al., 2010). Velos LTQ-orbitrap was also used to perform top-down MS and select ion monitoring (SIM) ETD MS/MS on each of these peaks to sequence the PTM sites on H3. Specifically from non-stimulated BMDMs, through multi-layer SIM ETD MS/MS ‘purification’ the pre-dominate species in Peak #2 was identified as a multiply modified form of H3.1 containing H3K4me2, K9me2, and K27me2. Also, an adjacent peak (Peak #1) at 14 Da upfield of Peak #2 contained a mixture with a population carrying an acetylation at lysine 9 (K9ac) along with H3K4me2 and K27me2. Notably, the relative K9ac abundance was increased under NL but decreased in TL BMDMs, whereas the opposite in K9me2 abundance was found. Nevertheless, all data identified the key PTM conversion at H3K9 with the TL-specific increase of K9me2 on H3. Because the methylation activity of G9a directly correlates with the accumulation of H3K9me2, combined MS results indicated that, specifically in LPS-tolerized macrophages, G9a promotes acetylation-to-methylation conversion at H3K9.

### Example 2

### G9a is Activated within T-Gene Chromatins During ET

The inflammation-phenotypic PTM change at H3K9 implied that the H3K9-targeting activity of G9a could be differentially regulated at T-gene promoters in TL versus NL cells. To clarify this, ChIP experiments were performed with anti-K9me2 antibody on the BMDM set under different inflammatory states. Coincident with the MS data, decreasing dimethylation and increasing acetylation of H3K9 were observed at both distal and proximal promoter regions of a T-gene (IL6) under NL. However, in ET macrophages, an increased H3K9me2 was observed at the same regions beyond TSS, indicating that the accumulation/occupancy of H3K9me2 on T-gene promoters during ET introduces a block for both transcription initiation and elongation of T-class genes. Similarly, in ChIP analysis with anti-G9a antibody, dramatically enhanced binding of G9a to the promoter region and the first intron was observed during ET, indicating that G9a is recruited to the cis-regulatory elements of IL6 and represses its expression. To clarify the role of G9a in ET-specific dimethylation of H3K9, prior to LPS stimulation BMDMs were treated with UNC0638 that inhibits the methylation activity of the enzyme but causes little change in its abundance (Vedadi et al., 2011). In ChIP analysis with anti-H3K9me2 antibody, greater decreases in H3K9me2 were observed in the inhibitor pre-treated TL cells compared with those under NL. Similar results were obtained from Raw264.7 cells with G9a knock-down, thus up-regulation of the methylation activity of G9a correlates with the gene-specific increase in H3K9me2.

### Example 3

### ChaC Enriches Active G9a and its Phenotypic Associates

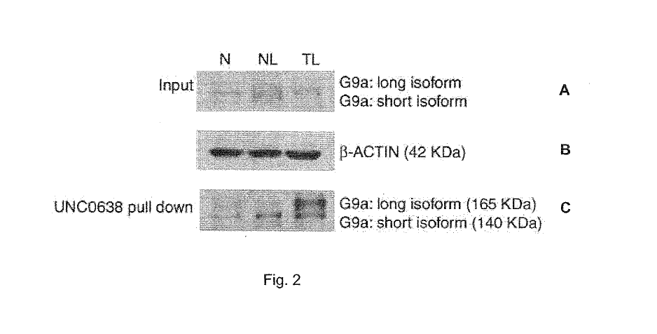

UNC0638 specifically binds to the enzymatically active form of G9a (Vedadi et al., 2011). The disclosed ChaC design was conceived to profile the inflammation-phenotypic G9a-interacting proteins within chromatin of differently inflamed macrophages. Moreover, as part of the ChaC design the incorporation of amino acid-coded mass tagging (AACT) as in-spectra markers in MS-based quantitative analysis (Chen et al., 2000; Zhu et al., 2002) provides for the ability to distinguish protein constituents in the G9a complexes pulled-down by UNC0638 respectively from primary BMDMs under different inflammatory states. First, a UNC0638 derivative, UNC2249, was synthesized with an aminopropyl linker for coupling UNC0638 to Sepharose beads that retained the same high affinity to G9a as UNC0638. As illustrated in FIG. 1, the UNC0638 beads were incubated with nuclear extract from equal amounts of BMDMs under either N (¹³C-Arg/Lys-labeled, medium), or NL (¹³C¹⁵N-Arg/Lys, heavy), or TL (unlabeled, light). While the level of G9a showed a slight decrease in TL cells compared with that in NL macrophages, significantly more G9a was pulled-down by UNC2249 from TL cells than from equal amounts of NL cells, indicating higher amounts of chronic-active G9a within the silenced chromatin under ET (FIG. 2). The pull-down proteins from each inflammatory state were mixed in equal proportion based on total protein mass and subjected to nanoLC-MS/MS analysis. In two biological replicates, 2021 proteins were quantified under NL or TL compared with their abundances in N cells; the ratios of NL/N and TL/N, respectively, represent the relative binding strengths of the corresponding proteins to G9a in either NL or TL compared with binding strength in non-stimulated cells. Thus, G9a was found to interact with different proteins, or the same proteins with different binding strengths, based on the inflammation-phenotypic methylation activity of G9a in differentially inflamed macrophages.

Then, the quantitative threshold that distinguishes ET-specific G9a-associating proteins was investigated. Scatter plots of the pull-down proteins with TL/N and NL/N ratio measured by AACT-based quantitative were developed. NL- or TL-specific interactors were then identified. First, log₂(TUN) or log₂(NUN) of G9a was found at 1.58 or −0.52, respectively, which was a similar trend with the inflammation-phenotypic changes in the methylation activity of G9a. Second, based on the ratios for multiple proteins known to co-exist with G9a in various complexes, such as HP1 (El Gazzar et al., (2008), GLP, HDAC1, HDAC2, LSD1/KDM1A, WIZ, SIN3A, and Brg1, the threshold to distinguish TL-specific G9a interactors was empirically determined as the borderline ratios with log₂(TUN)>0.82 and log₂(NUN)<0.68 or log₂(TL/NL)>1, i.e., the associations between G9a and these proteins are enhanced specifically in TL cells but weakened or non-specific under NL. Notably, in contrast to the relatively poor-correlated ratios of NL/N for the proteins quantified by biological replicates, the TL/N ratios of these proteins showed good reproducibility with correlation of linearity (R value at 0.82).

To validate the physiological accuracy of ChaC-identified proteins, an investigation was conducted to determine whether their recruitment onto T-gene promoters is truly dependent upon chronic-active G9a. Paired Raw264.7 cell lines were generated with either stable shRNA-mediated G9a knock-down (KD) or its wild-type counterpart Raw264.7 cells transfected with the empty vector (EV), from which the results are compared with the UNC0638-treated primary BMDMs under defined inflammatory conditions. First, given that HDAC1 is a shared component of multiple complexes pulled-down with G9a specifically during ET, ChIP-PCR was used to examine the inflammation-phenotypic binding of HDAC1 to a T-gene (IL6) promoter. Compared with wild-type Raw264.7 cells under NL, IL6-specific binding of HDAC1 was increased during ET, which was then diminished in either G9a-KD cells or UNC0638-treated BMDMs. Thus, the ET-specific recruitment of HDAC1 at the T-class promoter is dependent upon chronic-active G9a. Conversely, recruitment of G9a was affected by HDAC, as the abundance of G9a was reduced within the T-class promoters in the TL cells pre-treated by the HDAC inhibitor SAHA, implying a coordinated action between both histone-modifying enzymes when HDAC removes the acetyl group at H3K9 to facilitate accessibility of G9a. Meanwhile, SirT-1, a substrate of G9a (Moore et al., 2013), that plays a DE acetylation role during ET (Liu et al., 2011; Liu et al., 2012), was also quantified as a TL-specific G9a associator (1.1, −0.88), and showed a similar enrichment on IL6 as HDAC1, indicating both play the redundant role in K9 de-acetylation. Previous work showed the LPS-induced recruitment of Brg1, a key component of chromatin remodeling complex, on T-genes was attenuated under TL (Xie et al., 2013). However, in the G9a-depleted cells, recruitment of Brg1 was recovered on the IL6 distal promoter region, indicating that G9a plays a crucial role in determining Brg1 occupation within the T-gene chromatin while G9a co-exists with Brg1 in the SWI/SNF complexes. Notably, the consistent observations in G9a KD Raw264.7 cells versus UNC0638-treated BMDMs demonstrated the high specificity of UNC0638 in binding to the chronic-active G9a that coordinates assembly of a repressome within the silenced, LPS-tolerant chromatin.

### Example 4

### Chronic-Active G9a is at the Core of a Repressome Complex

Meeting the quantitative criteria above, 606 proteins were identified as TL-specific G9a interactors, which are predominantly clustered in functions such as DNA methylation, mRNA processing, transcriptional repression, and macromolecule metabolism. Further, by using STRING to explore the protein-protein interaction network, among these TL-specific G9a-interacting proteins, a few functional clusters that contain the intensive interactions were identified, indicating these canonical pathways/programs are interactive during ET. Multiple factors previously known as components of CoREST complex (Ooi et al., 2007), CtBP complex (Shintani et al., 2003), SWI/SNF chromatin remodeling complexes (CRCs) (Wilson et al., 2011), and NuRD complex (Ahringer, 2000) were also identified. For example, HDAC2, LSD1, Brg1, SIN3A, and G9a are the known components of CoREST complex, a transcriptional repressor module (Ooi et al., 2007). Simultaneously, a few proteins associated with carbohydrate metabolic processes, such as GRP78 and UGGG1, were identified, consistent with that CtBP is a NADH-dependent transcriptional repressor that links carbohydrate metabolism to epigenetic regulation by recruiting diverse histone-modifying complexes (Di et al., 2013). Further, WIZ, a known substrate of G9a, which links the G9a/GLP heteromeric complex to the CtBP co-repressor machinery (Ueda et al., 2006), was identified. Accordingly ChIP-PCR validated that recruitment of CtBP2 within the T-class promoter was G9a-dependent during ET. In the same pull-down complexome, multiple components/subunits of ATP-dependent CRCs and NuRD, were found to interact with chronic-active G9a. Given that the G9a-mediated methylation of MTA1, a key component of NuRD, is required for repression by NuRD (Nair et al., 2013), ChIP-PCR was also employed to investigate the G9a-dependent recruitment of the NuRD complex during ET. It was then observed that the enhanced TL-specific binding of MTA1 to T-class genes was diminished in either G9a-KD Raw cells or UNC0638-treated BMDMs, indicating the chronic-active G9a determines the stability of a functional NuRD repressor complex within T-chromatin.

To partly explore the functional implication of the TL-specific G9a interactions, the dataset of UNC2249/UNC0638 pull-down proteins from TL BMDMs was compared with the documented list of G9a non-histone substrates. 8 out of 16 proteins, including ACINUS, C/EBPt and WIZ that were the known G9a substrates, were found in the UNC2249 pull-down complex. Further, the TL-specific interactome constructed based on the UNC2249 pull-down data showed significant overlap with what was mapped based on the G9a-target Kme proteome (Moore et al., 2013; Islam et al., 2013), indicating that these proteins are the inflammation-phenotypic substrates of chronic-active G9a. Notably, although UNC0638 is a G9a substrate-competitive inhibitor, certain G9a substrates were pulled down probably due to their roles as the co-factors associating with other proteins that directly interact with the enzymatically active G9a, implicating that chronic-active G9a broadly facilitates the TL-specific complex assembly by modulating Kme-dependent protein-protein interactions. Meanwhile, many proteins previously unknown to associate with G9a were identified during ET, generating multiple hypotheses that extend current knowledge of the mechanisms involved in the transcriptional regulation of endotoxin tolerance.

### Example 5

### Chronic-Active G9a Interacts with Select HMTases During ET

For the first time, ChaC revealed an enhanced association between the H3K36-targeting HMTase NSD3 and G9a specifically in ET cells (based on scatter plot analysis of pull-down proteins as discussed above), which was validated by an in vitro co-immunoprecipitation experiment, implicating the cooperative action of both HMTases during ET. Further, to clarify whether the H3K36-targeting methylation activity of NSD3 depends on chronic-active G9a, the abundance of dimethylated H3K36 in either paired wild-type vs. G9a-KD Raw264.7 macrophages or non-treated vs. UNC0638-treated BMDMs was compared. Compared with its TL-specific increase in wild-type cells, the level of H3K36me2 decreased in either G9a-KD or UNC0638-treated cells (determined using gel electrophoresis and immunoblot analysis), indicating that both the methylation activity of NSD3 and the deposition of the K36me2 code on H3 are G9a-dependent during ET. Notably, the methylated level of H3K27 was also reduced in G9a-KD or UNC0638-treated cells in line with a report that G9a also targets H3K27 (Wu et al., 2011). Additionally, the TL-specific enrichment of NSD3 at the IL6 promoter was found dependent upon chronic-active G9a, revealing that chronic-active G9a also coordinates the combinatorial deposition of multiple Kme codes on H3 by recruiting NSD3 into T-chromatin. Further, a recent report indicated that both NSD3 and G9a participate in LSD2 complex for regulating transcription elongation by modulating intragenic H3K4me2 (Fang et al., 2010), supporting the disclosed findings of the co-existence of both G9a and NSD3 in the TL-specific complexome. Meanwhile, ASH2I, another HMTase that catalyzes H3K4 tri-methylation for transcriptional activation and is also a core member of the transcription-initiating COMPASS/COMPASS-like complexes (Smith et al., 2011), was identified along with other members of COMPASS, WDR82 and CXXC1. The enhanced interaction between G9a and ASH2I in ET macrophages was also validated by co-transfection and co-IP experiments. Further, even in the LPS-tolerant macrophages, G9a showed a higher affinity to the peptide containing H3K4me2 than to its unmodified counterpart, while the H3K4me2/3 level was little changed across all inflammatory conditions. Thus, following acute stimulation G9a may become chronically active by first docking on ASH2I-target H3K4me3 deposited in the transcriptional-active chromatin associated with T-genes.

### Example 6

### Chronic-Active G9a Promotes Gene-Specific Co-Repression

From the silenced chromatin of LPS-tolerized BMDMs, it was also found that UNC2249/UNC0638 selectively enriched/pulled-down a cluster of low-abundance transcriptional factors (TFs) or DNA-binding proteins. Notably, both transcriptional repressive subunits of the NF-κB family, RelB and C-Rel were identified specifically in the chronic-active G9a complexome, while other NF-κB subunits including p65 and p100/p105 were identified with random, lower TL/N. Meanwhile, ChIP experiments showed NL-specific recruitment of p65 to T-genes, indicating dissociation of the inflammation-activating NF-κB subunit(s) from the silenced T-gene chromatin, while repressive NF-κB was selectively recruited into the TL-specific complexome. Additionally, a transcriptional repressor YinYang1(YY1) that was implicated in mediating transcriptional repression by interacting with either CtBP (Srinivasan et al., 2004), or Sin3, or HDACs (Lu et al., 2011), was also identified as a TL-specific complexome component. Recent work (Siednienko et al., 2011) indicated that NF-κB-dependent increased YY1 expression negatively regulates IFNβ production, possibly mediated by C-Rel and RelB. Importantly, here, YY1 was found together with these co-repressor complex components or transcriptional repressors during ET. Accordingly, existence of YY1 at T-gene (IL6) promoters was found dependent upon G9a in ET macrophages, indicating that chronic-activated G9a facilitates the simultaneous access of select TFs to T-gene promoters during ET.

To determine the impact of the co-recruitment of combinatorial TFs with G9a on gene-specific expression during ET, AACT-based quantitative proteomics (Chen et al., 2000; Zhu et al., 2002) was conducted on paired wild-type and G9a-KD Raw264.7 macrophages under TL to identify/profile the global, differential proteome expression caused by G9a depletion. In LPS-tolerant macrophages, 421 out of 5,393 quantifiable proteins showed G9a-repressed expression by more than 2-fold; statistically over-represented in GO function clusters for immune response, apoptosis, and cell proliferation. By using Ingenuity Pathway Analysis a well-connected gene regulatory network was identified among 198 G9a-repressing proteins that were previously known gene products regulated by either one or several of select TFs. Many of these proteins at the network nodes are regulated by multiple TFs, indicating that these TFs that are selectively recruited with chronic-active G9a work in concert to co-regulate their target genes. These data reveal that chronic-active G9a extends its repressive role in the chromatin-associated, gene-specific regulation of ET by coordinating the co-regulatory action of select transcriptional repressors.

### Example 7

### Chronic-Active G9a Interacts with Interferon-Inducible Complexes

Strikingly, a cluster of IFN-inducible proteins including interferon (IFN)-inducible protein 35 (IFI35) and N-myc-interactor (Nmi) was identified as IL-specific, G9a-interacting proteins. Both the involvement of G9a in IFN signaling (Gyory et al., 2004) and H3K9me2 as an epigenetic signature of IFN/cytokine signaling (Fang et al., 2012) were suggested by this finding. Therefore, it was reasoned that chronic-active G9a interacts with this cluster of proteins to extend its function in under-characterized regulatory pathway(s) for gene-specific regulation during ET. Except for its role in the transcription repression mediated by c-Myc oncogene (Herkert et al., 2010), the function of IFI35 in inflammation control is ambiguous. First, it was found in the disclosed studies that the expression of IFI35 remained LPS-inducible even under ET, showing the typical feature of non-tolerizable (NT)-class genes (Foster et al., 2007). Meanwhile, G9a-IFI35 association was significantly enhanced during ET. Further, the catalytic SET domain of G9a was found directly involved in the ET-specific G9a-IFI35 interaction, suggesting a determining role of G9a methylation activity in coordinating the interferon-inducible complex specifically during ET.

Except for c-Myc, other proteins in the complexes of STATs and Myc proteins (Bannasch et al., 1999) were all identified as TL-specific G9a-interacting partners. To test the conjecture that failure to detect c-Myc was due to its high-turnover rate, the in vitro, inflammation-phenotypic interaction of G9a with c-Myc as well as with other proteins was examined. Consistent with TL-specific G9a-c-Myc interaction, all four proteins enhanced their association in the same complex immunoprecipitated from the LPS-tolerant cells co-transfected with four epitope-tagged constructs respectively expressing IFI35, G9a, c-Myc, Nmi. Further, these associations were promoted by both G9a and H3K9me2, as the complex involving all components was pulled-down only from the co-transfected cells under TL using a H3K9me2-containing peptide. Thus, both chronic-active G9a and H3K9me2 are required for the integrity of IFI35/c-Myc/Nmi complex that specifically recognizes/docks on H3K9me2 during ET.

It was further investigated whether c-Myc impacts the function of G9a during ET by measuring the methylation activity of G9a with or without over-expressing c-Myc. Epitope-tagged G9a were transfected or both c-Myc and G9a were co-transfected into the stable TLR4-expressing cells under TL. With a H3 N-terminal peptide as the substrate, HMTase activity increased with the co-expression of both proteins, indicating that c-Myc is a part of the complex involving chronic-active G9a. The impact of this G9a/K9me2-dependent complex on NE-KB-dependent transcription was then investigated. An NF-κB-target luciferase reporter was co-transfected with the expression vector(s) of either G9a, or c-Myc, or both. As shown via luciferase assays, LPS-induced NF-κB activity decreased with increasing expression of either G9a or c-Myc, while IFI35 and Nmi had little effect on G9a/c-Myc-associated suppression of NF-κB activity, indicating that c-Myc and chronic-active G9a work synergistically to suppress pro-inflammatory NF-κB activity during ET. Also, this G9a-dependent suppression of NF-κB activity is readily interpreted by the observation that G9a-dependent existence of RelB, the repressive subunit of NF-κB, in the T-gene nucleosome during ET.

### Example 8

### c-Myc Regulates G9a-Dependent Gene-Specific Transcription

Myc broadly targets more than 1,500 genes involving in diverse biological processes (Dang et al., 2006). To uncover G9a-dependent, c-Myc-regulated pathways/processes, ChIP-seq experiments were performed using anti-c-Myc antibody on paired wild-type and G9a-KD Raw264.7 cells under TL. Comparative genome-wide mapping of c-Myc binding loci was obtained to identify the G9a-dependent occupancy of select c-Myc-regulated genes. Using gent ontology biological process analysis, G9a knock-down led to decreased binding of c-Myc to 1132 genes, including a number of T-genes such as IL10, Lipg, Itga4 and apoptosis-related genes including Bax and Bcl2l2-Pabpn1. For instance, the enrichment of c-Myc at the IL10 distal promoter was found in wild-type but not in G9a-KD cells. No peak was found in the proximal promoter of IL10 in either wild-type or G9a-KD cells, consistent with the results from ChIP-PCR validation. Notably, 38% of the G9a-dependent, c-Myc binding genes identified by ChIP-seq are c-Myc-regulated genes found in fibroblasts and B-lymphoid tumor cells (Perna et al., 2012; Zeller et al., 2006) indicating that c-Myc also contributes to ET through regulating select genes in the G9a-dependent manner. Further, ChIP-qPCR validated the dual-dependence of other known T-class or NT-class genes. For example, c-Myc showed enhanced binding to the loci of IL6 specifically under TL, which was diminished consistently in either UNC0638-treated BMDMs or G9a-KD Raw264.7 cells. c-Myc binding to the NT-genes was not affected by either the inhibitor or G9a-KD, all demonstrating that the TL-specific recruitment/accessibility of c-Myc to T-gene promoters is dependent upon chronic-active G9a. As well, along with additional recruitment of c-Myc to IL6, IP-10, and p21 (the universal cell cycle inhibitor p21^{cip}) (Wong et al., 2012) in the wild-type cells under TL compared with NL, siRNA-mediated knock-down of G9a or UNC0638 inhibition of chronic-active G9a led to reduced c-Myc binding to these genes. Given the pro-inflammatory nature of IL6, this result suggested that the G9a-dependent binding of c-Myc to T-gene promoters is responsible for the suppression of this class of the genes during ET. Further, given that both IP-10 and p21 are known c-Myc-regulated genes involved in cell survival, it was concluded that chronic-active G9a extends its epigenetic repressive role in cell fate decision by regulating the transactivation activity of c-Myc during the inflammation control of ET.

### Example 9

### Chronic-Active G9a Inhibits c-Myc Degradation During ET

Given the newly characterized, broad effect of c-Myc on transcriptional co-regulation, as disclosed herein, it was next investigated how chronic-active G9a regulates c-Myc during ET. The inflammation-phenotypic expression of c-Myc in wild-type versus G9a-KD Raw264.7 cells was first investigated. For wild-type cells, compared with its abundance under N or NL, slightly less c-Myc was observed under TL. Although little change was found for both wild-type and G9a-KD cells under N or NL, the abundance of c-Myc was substantially lower in the TL G9a-KD cells, suggesting the role of chronic-active G9a in maintaining the abundance of c-Myc in LPS-tolerant macrophages. It was then determined at what levels G9a regulates c-Myc stability. First, RT-PCR showed that c-Myc mRNA was elevated in G9a-depleted cells, suggesting that G9a suppresses c-Myc transcription. Second, the rate of c-Myc decay following inhibition of protein synthesis with cycloheximide (CHX) was about four times slower in the TL wild-type cells than in the G9a KD counterpart. Also, more c-Myc was detected with increasing expression of G9a, indicating that chronic-active G9a stabilizes c-Myc at T-gene promoters by inhibiting c-Myc degradation during ET. Further, we respectively pre-treated either wild-type or G9a-KD cells with the proteasome inhibitor MG-132, which allowed analysis of the time-dependent translation of c-Myc mRNA when protein degradation was blocked. In contrast to the time-dependent, progressive accumulation of c-Myc in G9a-KD cells, the MG-132-induced c-Myc was actually reduced with a longer post-treatment time in the presence of G9a, indicating that G9a also attenuates c-Myc translation, consistent with the TL-specific decrease of c-Myc in the wild-type cells. Thus, G9a-dependent inhibition of c-Myc degradation at the post-translational level is the predominate process that determines the stability and then the accessibility of c-Myc onto T-gene promoters.

### Example 10

### G9a Suppresses Cell Response and Gene-Specific Secretion

To further clarify the impact of G9a-mediated stabilization of c-Myc on cell fate decision during ET, wild-type and G9a-KD cells were pre-treated with UNC0638 and chronically stimulated them with LPS. While little effect of UNC0638 treatment was observed for the non-treated wild-type cells, the relatively high percentage of survived cells under TL was significantly reduced in cells pre-treated with UNC0638. Thus, UNC0638 could re-sensitize ET macrophages and promote their apoptosis by inhibiting chronic-active G9a, revealing that c-Myc plays a cooperative role with TL-specific G9a complexome, the IFN signaling cluster in particular, in regulating endotoxin tolerance.

The impact of chronic-active G9a on gene-specific transcription and translation was further investigated. First, a label-free quantitative proteomic method for profiling BMDM secretome (Meissner et al., 2013) was employed to identify the proteins differentially secreted from the BMDMs under N, NL, TL without or with UNC0638 treatment respectively. As a result, the secretion levels of 45% of a total of 992 identified proteins showing repressed releases was found increasing in the UNC0638-pre-treated BMDMs under TL. RT-PCR analysis of some of these T-genes or 18 out of 31 selected genes also showed the similar trend of G9a-dependent changes, all indicating that chronic-active G9a broadly affects on the endotoxin-induced transcription and translation of select pro-inflammatory/T-class genes.

### Discussion of Examples 1-10

Because protein complexes are immunoprecipitated based on the abundance and not the activity of the bait protein (Malovannaya et al., 2011), conventional antibody-dependent approaches for interactome screening are less sensitive and/or accurate in distinguishing in situ functional interactions. Disclosed herein is a breakthrough activity-based ChaC methodology that can reveal the inflammation-phenotypic, functional constituents of the chromatin writer complexome that regulates gene-specific transcription. Unlike most ABPP inhibitors that bind to the active site of their target enzymes, disclosed ChaC chemoprobes are substrate-competitive ligands, so that the G9a-scaffolding function to accommodate the functionally relevant protein interactions is preserved. Further, because of its high-specificity in binding to active G9a, both antibody specificity and cross-activity are no longer the concerns.

Firstly, the characterization of HDAC1, SirT-1, and Brg1 as the TL-specific G9a-associating partners, as disclosed herein, reveals that chronic-active G9a holds up particular chromatin remodeling complexes within the silenced chromatin, and, in parallel, both HDAC1 (Shintani et al., 2003) and SirT-1 are recruited to chromatin and then work in concert with chronic-active G9a to promote the phenotypic PTM conversion at H3K9. Secondly, in the TL-specific complexome, G9a interacts with select writer(s) or eraser(s) of these methyl-lysine sites, leading to concerted changes of multiple methyl-lysine marks. The constitutive interaction between ASH2I and G9a, as revealed by data collected in the disclosed examples, provided an interpretation for the previous finding that G9a targets H3K9 at the euchromatic regions demarcated by K4me2/3 where G9a is first recruited to the transcriptional-activated regions to later suppress transcription of T-genes (Tachibana et al., 2002). H3K4me2/3 plays an auxiliary role, which is maintained by the G9a-ASH2I interaction. Here, the combined results provide a systems view that, during ET, chronic-active G9a coordinates deposition of combinatorial Kme sites on H3 known to play either an individual or combinatorial role in gene suppression (Kouzarides, 2007) to synergistically maintain a repressive chromatin landscape at T-gene promoters.

In tumor cells, c-Myc is an oncogenic transcriptional activator (Dang et al., 2006) that preferentially targets genes enriched with the transcriptional-active codes, such as H3K4me2/3 and H3K9ac, through interaction with a variety of histone acetyltransferase (HAT)-associated protein complexes where c-Myc is also a substrate of defined HATs and can be stabilized by acetylation (Patel et al., 2004). Conversely, little is known about how the transcriptional activity of c-Myc is regulated during inflammation control. The instant disclosure is the first to find that, specifically in ET macrophages, c-Myc interacts with G9a in an IFI35/c-Myc/Nmi complex that can dock on the transcriptional repressive mark of H3K9me2. Multiple c-Myc-regulated genes were also identified in the G9a-dependent co-regulatory network involving a combinatorial set of transcriptional repressors, indicating that c-Myc is a major component of the G9a repressome. Further, given that c-Myc is expressed ubiquitously, and its stability is essential for its transcriptional activity, the disclosed finding that chronic-active G9a stabilizes c-Myc within promoters of select genes implicates the G9a-dependent role of c-Myc in transcriptional repression during ET. Therefore, c-Myc is a dual-functional transcriptional factor, alternatively acting as either activator or repressor based on its epigenetic environment involving interacting histone codes and select histone-modifying enzymes. During ET c-Myc becomes a G9a/H3K9me2-dependent transcriptional repressor in a similar way as it acts as a HAT/K9ac-dependent transcriptional activator. This conclusion is also supported by reports that c-Myc plays a regulatory role in anti-inflammatory macrophages⁵⁴ and it interacts with a G9a-associating factor Gfi-1 at the p21-promoter region to epigenetically repress p21 (Duan et al., 2005), indicating that the epigenetic mechanisms dictate the phenotypic binding of Myc to select target genes. Further, global profiling of G9a-regulated protein expression together with ChIP-seq to identify the regulatory DNA elements reveal select genes showing the dual-dependence on both G9a and c-Myc during the ET phenotype. These genes mostly fall into the over-represented functional categories associated with immune or inflammatory response, and apoptosis, demonstrating exactly how G9a coordinates the gene-specific regulation of chronic inflammation through the methylation-activity-based complexome assembly.

Previous evidence from independent studies suggests that, in endotoxin-tolerant macrophages, nucleosomes are repositioned from permissive to repressive locations, and both promoter chromatin remodeling and NE-KB transactivation are disrupted (Chan et al., 2005). The disclosed repressome findings now provide the molecular illustration that the specificity of the inflammation-phenotypic chromatin reprogramming is controlled by particular transcription factors and co-factors that are selectively recruited: during ET, select transcriptional repressors including RelB, YY1, c-Myc were first recruited into the T-gene chromatin (Chen et al., 2009; Tong et al., 2013 to replace the transcriptional activators such as RelA/p65, and then interact with the chronic-activated G9a which in turn stabilizes the binding of these factors to select classes of the genes. This idea is consistent with the previously reported correlation between RelB expression and the induction of repressive nucleosome positioning (El Gazzar et al., 2010) as well as the in vitro interaction between GST-tagged G9a and RelB during ET (Chen et al., 2009). Meanwhile, the major factor complexes associated with chromatin plasticity and mobilization including NuRD and SWI/SNF complexes were simultaneously identified in the G9a repressome, indicating that chronic-active G9a interacts with these complexes to integrate activities of nucleosome positioning, ATP-dependent chromatin remodeling, and transcriptional repressors. Also supported by comparative proteomic experiments on paired wild-type and G9a-KD macrophages, in which global protein products of G9a-silenced genes were identified, these findings support the conclusion that chronic-active G9a participates in the co-regulation by select transcriptional repressors in cross talk that synergistically pronounce the gene-specific repression.

On a systems view, the instant disclosure is the first to demonstrate that as the writer of the chromatin repressive mark H3K9me2, in a methylation-activity-dependent manner, G9a couples select biological pathways/programs across multi-phases of transcription including DNA binding, nucleosome localization, and transcription factor recruitment to the gene-specific epigenetic regulation by recruiting specific complexes to the chromatin of T-genes, which systematically define a transcriptionally silenced chromatin. Technically, the disclosed ChaC approach can generate activity-based interactome data in a physiologically accurate manner and is generically applicable to dissect a variety of phenotypic chromatin architectures in the context of a functional protein complexome.

### REFERENCES

1. Coussens, L. M. & Werb, Z. Inflammation and cancer. Nature 420, 860-867 (2002).
2. Clevers, H. At the crossroads of inflammation and cancer. Cell 118, 671-674 (2004).
3. Kanterman, J., Sade-Feldman, M. & Baniyash, M. New insights into chronic inflammation-induced immunosuppression. Semin Cancer Biol 22, 307-318 (2012).
4. Biswas, S. K. & Lopez-Collazo, E. Endotoxin tolerance: new mechanisms, molecules and clinical significance. Trends Immunol 30, 475-487 (2009).
5. Foster, S. L. & Medzhitov, R. Gene-specific control of the TLR-induced inflammatory response. Clinical immunology (Orlando, Fla. 130, 7-15 (2009).
6. Foster, S. L., Hargreaves, D. C. & Medzhitov, R. Gene-specific control of inflammation by TLR-induced chromatin modifications. Nature 447, 972-978 (2007).
7. Xie, L. et al. Protein phosphatase 2A catalytic subunit alpha plays a MyD88-dependent, central role in the gene-specific regulation of endotoxin tolerance. Cell reports 3, 678-688 (2013).
8. Johnson, D. G. & Dent, S. Y. Chromatin: receiver and quarterback for cellular signals. Cell 152, 685-689 (2013).
9. Badeaux, A. I. & Shi, Y. Emerging roles for chromatin as a signal integration and storage platform. Nat Rev Mol Cell Biol 14, 211-224 (2013).
10. El Gazzar, M. et al. G9a and HP1 couple histone and DNA methylation to TNFalpha transcription silencing during endotoxin tolerance. The Journal of biological chemistry 283, 32198-32208 (2008).
11. Tachibana, M. et al. G9a histone methyltransferase plays a dominant role in euchromatic histone H3 lysine 9 methylation and is essential for early embryogenesis. Genes & development 16, 1779-1791 (2002).
12. Tachibana, M. et al. Histone methyltransferases G9a and GLP form heteromeric complexes and are both crucial for methylation of euchromatin at H3-K9. Genes & development 19, 815-826 (2005).
13. Ooi, L. & Wood, I. C. Chromatin crosstalk in development and disease: lessons from REST. Nat Rev Genet 8, 544-554 (2007).
14. Shintani, S. et al. Up-regulation of DNA-dependent protein kinase correlates with radiation resistance in oral squamous cell carcinoma. Cancer Sci 94, 894-900 (2003).
15. Wilson, B. G. & Roberts, C. W. SWI/SNF nucleosome remodellers and cancer. Nature reviews. Cancer 11, 481-492 (2011).
16. Ahringer, J. NuRD and SIN3 histone deacetylase complexes in development. Trends Genet 16, 351-356 (2000).
17. Chen, X., El Gazzar, M., Yoza, B. K. & McCall, C. E. The NF-kappaB factor RelB and histone H3 lysine methyltransferase G9a directly interact to generate epigenetic silencing in endotoxin tolerance. J Biol Chem 284, 27857-27865 (2009).
18. Moore, K. E. et al. A general molecular affinity strategy for global detection and proteomic analysis of lysine methylation. Molecular cell 50, 444-456 (2013).
19. Malovannaya, A. et al. Analysis of the human endogenous coregulator complexome. Cell 145, 787-799 (2011).
20. Cravatt, B. F., Wright, A. T. & Kozarich, J. W. Activity-based protein profiling: from enzyme chemistry to proteomic chemistry. Annu Rev Biochem 77, 383-414 (2008).
21. Li, N. et al. Relative quantification of proteasome activity by activity-based protein profiling and LC-MS/MS. Nat Protoc 8, 1155-1168 (2013).
22. Cole, P. A. Chemical probes for histone-modifying enzymes. Nature chemical biology 4, 590-597 (2008).
23. Hwang, Y. et al. A selective chemical probe for coenzyme A-requiring enzymes. Angew Chem Int Ed Engl 46, 7621-7624 (2007).
24. Yu, M., de Carvalho, L. P., Sun, G. & Blanchard, J. S. Activity-based substrate profiling for Gcn5-related N-acetyltransferases: the use of chloroacetyl-coenzyme A to identify protein substrates. J Am Chem Soc 128, 15356-15357 (2006).
25. Salisbury, C. M. & Cravatt, B. F. Optimization of activity-based probes for proteomic profiling of histone deacetylase complexes. J Am Chem Soc 130, 2184-2194 (2008).
26. Vedadi, M. et al. A chemical probe selectively inhibits G9a and GLP methyltransferase activity in cells. Nat Chem Biol 7, 566-574 (2011).
27. Kagale, S., Links, M. G. & Rozwadowski, K. Genome-wide analysis of ethylene-responsive element binding factor-associated amphiphilic repression motif-containing transcriptional regulators in Arabidopsis. Plant Physiol 152, 1109-1134 (2010).
28. Zhao, S. et al. Regulation of cellular metabolism by protein lysine acetylation. Science 327, 1000-1004 (2010).
29. Chen, X., Smith, L. M. & Bradbury, E. M. Site-specific mass tagging with stable isotopes in proteins for accurate and efficient protein identification. Analytical chemistry 72, 1134-1143 (2000).
30. Zhu, H., Pan, S., Gu, S., Bradbury, E. M. & Chen, X. Amino acid residue specific stable isotope labeling for quantitative proteomics. Rapid Commun Mass Spectrom 16, 2115-2123 (2002).
31. Liu, T. F., Yoza, B. K., El Gazzar, M., Vachharajani, V. T. & McCall, C. E. NAD+-dependent SIRT1 deacetylase participates in epigenetic reprogramming during endotoxin tolerance. The Journal of biological chemistry 286, 9856-9864 (2011).
32. Liu, T. F., Vachharajani, V. T., Yoza, B. K. & McCall, C. E. NAD+-dependent sirtuin 1 and 6 proteins coordinate a switch from glucose to fatty acid oxidation during the acute inflammatory response. The Journal of biological chemistry 287, 25758-25769 (2012).
33. Di, L. J. et al. Genome-wide profiles of CtBP link metabolism with genome stability and epithelial reprogramming in breast cancer. Nature communications 4, 1449 (2013).
34. Ueda, J., Tachibana, M., Ikura, T. & Shinkai, Y. Zinc finger protein Wiz links G9a/GLP histone methyltransferases to the co-repressor molecule CtBP. The Journal of biological chemistry 281, 20120-20128 (2006).
35. Nair, S. S., Li, D. Q. & Kumar, R. A Core Chromatin Remodeling Factor Instructs Global Chromatin Signaling through Multivalent Reading of Nucleosome Codes. Molecular cell (2013).
36. Islam, K. et al. Defining efficient enzyme-cofactor pairs for bioorthogonal profiling of protein methylation. Proceedings of the National Academy of Sciences of the United States of America 110, 16778-16783 (2013).
37. Wu, H. et al. Histone methyltransferase G9a contributes to H3K27 methylation in vivo. Cell research 21, 365-367 (2011).
38. Fang, R. et al. Human LSD2/KDM1b/AOF1 regulates gene transcription by modulating intragenic H3K4me2 methylation. Molecular cell 39, 222-233 (2010).
39. Smith, E., Lin, C. & Shilatifard, A. The super elongation complex (SEC) and MLL in development and disease. Genes & development 25, 661-672 (2011).
40. Srinivasan, L. & Atchison, M. L. YY1 DNA binding and PcG recruitment requires CtBP. Genes & development 18, 2596-2601 (2004).
41. Lu, P. et al. The developmental regulator protein Gon4I associates with protein YY1, co-repressor Sin3a, and histone deacetylase 1 and mediates transcriptional repression. The Journal of biological chemistry 286, 18311-18319 (2011).
42. Siednienko, J. et al. Nuclear factor kappaB subunits RelB and cRel negatively regulate Toll-like receptor 3-mediated beta-interferon production via induction of transcriptional repressor protein YY1. The Journal of biological chemistry 286, 44750-44763 (2011).
43. Gyory, I., Wu, J., Fejer, G., Seto, E. & Wright, K. L. PRDI-BF1 recruits the histone H3 methyltransferase G9a in transcriptional silencing. Nature immunology 5, 299-308 (2004).
44. Fang, T. C. et al. Histone H3 lysine 9 di-methylation as an epigenetic signature of the interferon response. J Exp Med 209, 661-669 (2012).
45. Herkert, B. & Eilers, M. Transcriptional repression: the dark side of myc. Genes Cancer 1, 580-586 (2010).
46. Bannasch, D., Weis, I. & Schwab, M. Nmi protein interacts with regions that differ between MycN and Myc and is localized in the cytoplasm of neuroblastoma cells in contrast to nuclear MycN. Oncogene 18, 6810-6817 (1999).
47. Dang, C. V. et al. The c-Myc target gene network. Seminars in cancer biology 16, 253-264 (2006).
48. Perna, D. et al. Genome-wide mapping of Myc binding and gene regulation in serum-stimulated fibroblasts. Oncogene 31, 1695-1709 (2012).
49. Zeller, K. I. et al. Global mapping of c-Myc binding sites and target gene networks in human B cells. Proceedings of the National Academy of Sciences of the United States of America 103, 17834-17839 (2006).
50. Wong, P. P. et al. Histone demethylase KDM5B collaborates with TFAP2C and Myc to repress the cell cycle inhibitor p21(cip) (CDKN1A). Molecular and cellular biology 32, 1633-1644 (2012).
51. Meissner, F., Scheltema, R. A., Mollenkopf, H. J. & Mann, M. Direct proteomic quantification of the secretome of activated immune cells. Science (New York, N.Y. 340, 475-478 (2013).
52. Kouzarides, T. Chromatin modifications and their function. Cell 128, 693-705 (2007).
53. Patel, J. H. et al. The c-MYC oncoprotein is a substrate of the acetyltransferases hGCN5/PCAF and TIP60. Mol Cell Biol 24, 10826-10834 (2004).
54. Pello, O. M. et al. Role of c-MYC in alternative activation of human macrophages and tumor-associated macrophage biology. Blood 119, 411-421 (2012).
55. Duan, Z., Zarebski, A., Montoya-Durango, D., Grimes, H. L. & Horwitz, M. Gfi1 coordinates epigenetic repression of p21Cip/WAF1 by recruitment of histone lysine methyltransferase G9a and histone deacetylase 1. Molecular and cellular biology 25, 10338-10351 (2005).
56. Chan, C., Li, L., McCall, C. E. & Yoza, B. K. Endotoxin tolerance disrupts chromatin remodeling and NF-kappaB transactivation at the IL-1beta promoter. J Immunol 175, 461-468 (2005).
57. Tong, X. et al. Recruitment of histone methyltransferase G9a mediates transcriptional repression of Fgf21 gene by E4BP4 protein. The Journal of biological chemistry 288, 5417-5425 (2013).
58. El Gazzar, M., Liu, T., Yoza, B. K. & McCall, C. E. Dynamic and selective nucleosome repositioning during endotoxin tolerance. The Journal of biological chemistry 285, 1259-1271 (2010).
59. Gardner, K. E., Zhou, L., Parra, M. A., Chen, X. & Strahl, B. D. Identification of lysine 37 of histone H2B as a novel site of methylation. PLoS One 6, e16244 (2011).
60. Syka, J. E., Coon, J. J., Schroeder, M. J., Shabanowitz, J. & Hunt, D. F. Peptide and protein sequence analysis by electron transfer dissociation mass spectrometry. Proceedings of the National Academy of Sciences of the United States of America 101, 9528-9533 (2004).
61. Tian, Z. et al. Enhanced top-down characterization of histone post-translational modifications. Genome Biol 13, R86 (2012).
62. Wysocka, J. Identifying novel proteins recognizing histone modifications using peptide pull-down assay. Methods 40, 339-343 (2006).
63. Cox, J. et al. Andromeda: a peptide search engine integrated into the MaxQuant environment. Journal of proteome research 10, 1794-1805 (2011).
64. Cox, J. & Mann, M. Quantitative, high-resolution proteomics for data-driven systems biology. Annual review of biochemistry 80, 273-299 (2011).
65. Cox, J. & Mann, M. MaxQuant enables high peptide identification rates, individualized p.p.b.-range mass accuracies and proteome-wide protein quantification. Nature biotechnology 26, 1367-1372 (2008).
66. Gunawardena, H. P. et al. Comparison of the membrane proteome of virulent Mycobacterium tuberculosis and the attenuated Mycobacterium bovis BCG vaccine strain by label-free quantitative proteomics. Journal of proteome research 12, 5463-5474 (2013).
67. Gunawardena, H. P. et al. Unambiguous characterization of site-specific phosphorylation of leucine-rich repeat Fli-I-interacting protein 2 (LRRFIP2) in Toll-like receptor 4 (TLR4)-mediated signaling. The Journal of biological chemistry 286, 10897-10910 (2011).
68. Porro, A. & Perini, G. In vivo dual cross-linking chromatin immunoprecipitation: detecting chromatin proteins not directly bound to DNA. The Epigenome-Network of Excellence: Epigenetics protocols database (2007).
69. Langmead, B. & Salzberg, S. L. Fast gapped-read alignment with Bowtie 2. Nat Methods 9, 357-359 (2012).
70. Zhang, Y. et al. Model-based analysis of ChIP-Seq (MACS). Genome Biol 9, R137 (2008).

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

It will be understood that various details of the presently disclosed subject matter may be changed without departing from the scope of the presently disclosed subject matter. Furthermore, the foregoing description is for the purpose of illustration only, and not for the purpose of limitation.

1. A method for identifying a disease-related component of a gene-specific chromatin regulatory protein complex, comprising:
providing a sample to be assayed;
contacting the sample with a chemoprobe, wherein the chemoprobe is substrate-competitive and selectively binds to an enzymatically active enzyme associated with a gene-specific chromatin regulatory protein complex; and
detecting a gene-specific chromatin regulatory protein complex present in the sample using the chemoprobe based on the enzymatically active enzyme associated with the gene-specific chromatin complex.

2. The method of claim 1, wherein detecting the gene-specific chromatin regulatory protein complex comprises identifying one or more components of the gene-specific chromatin regulatory protein complex.

3. The method of claim 1, wherein the enzyme associated with the gene-specific chromatin regulatory protein complex comprises a histone-modifying enzyme, a histone post-translational modification (PTM)-reading protein, a co-regulatory protein complex, and/or a transcriptional factor.

4. The method of claim 1, wherein the chemoprobe comprises an inhibitor of a histone-modifying enzyme or a histone PTM reader domain, optionally wherein the histone-modifying enzyme is selected from the group consisting of G9a and Ezh2, and optionally wherein the histone PTM reader domain is selected from the group consisting of a bromodomain (BRD) antagonist or acetyl-lysine (Kac).

5. The method of claim 1, wherein the chemoprobe is selected from the group consisting of UNC0638, UNC1999 and I-BET, optionally wherein UNC0638 is immobilized on Sepharose beads (UNC2249), optionally wherein UNC0638 is biotinylated (UNC0965), optionally wherein UNC1999 is biotinylated (UNC2399), and/or optionally wherein I-BET is biotinylated (UNC3660A).

6. The method of claim 5, wherein UNC0638 and/or UNC2249 and/or UNC0965 comprises a substrate-competitive inhibitor that selectively binds enzymatically active G9a, wherein UNC1999 and/or UNC2399 comprises a substrate-competitive inhibitor that selectively binds enzymatically active Ezh2, wherein UNC3660A comprises a substrate-competitive inhibitor that selectively binds enzymatically active BRD.

7. The method of claim 1, wherein the enzymatically active enzyme associated with a gene-specific chromatin regulatory protein complex defines a transcriptional activity of a chromatin associated with a class of genes.

8. The method of claim 1, wherein the isolated chromatin regulatory protein complex comprises a functional chromatin-modifying complex within chromatin associated with select genes.

9. The method of claim 8, wherein the functional chromatin complex reveals how an activity-based protein complexome is assembled within the chromatin of defined transcriptional activity, and/or where it is localized in the genome.

10. The method of claim 1, comprising contacting the sample with an affinity-tagged chemoprobe that selectively binds a chromatin modifier, a chromatin eraser, or a chromatin reader, optionally wherein the chromatin modifier is selected from G9a and Ezh2, and optionally wherein the chromatin reader is BRD.

11. The method of claim 10, comprising contacting the sample with two biotinylated chemoprobes, wherein a first chemoprobe selectively binds G9a, and wherein a second chemoprobe selectively binds a BRD, wherein corresponding protein complexes from transcriptional active genes or transcriptional repressive genes, respectively, can be isolated.

12. The method of claim 1, wherein the presence of a gene-specific chromatin regulatory protein complex in the sample is indicative of a disease phenotype.

13. The method of claim 12, wherein the disease phenotype comprises a chronic inflammation-associated disease phenotype.

14. The method of claim 12, wherein the presence of the gene-specific chromatin regulatory protein complex that is indicative of a disease phenotype comprises one or more biomarkers.

15. The method of claim 1, wherein the gene-specific chromatin regulatory protein complex is associated with disease-related genes selected from the group consisting of disease-causing, disease-suppressing, and tumor-suppressing genes.

16. The method of claim 1, wherein the chemoprobe is immobilized on a substrate.

17. The method of claim 16, wherein the substrate comprises a bead.

18. The method of claim 16, further comprising immobilizing the chemoprobe in a pipette tip or multi-well plate.

19. The method of claim 1, wherein the chemoprobe is affinity tagged, optionally wherein the affinity tag comprises biotin.

20. The method of claim 1, further comprising sequencing of one or more components of the gene-specific chromatin regulatory protein complex for biomarker identification.

21. The method of claim 1, wherein the sample is selected from the group consisting tissue, blood and plasma.

22. The method of claim 1, further comprising identifying a gene-specific binding of a transcriptional factor.

23. The method of claim 1, further comprising identifying a co-regulator network.

24. A high-throughput method for screening for a disease biomarker using chromatin activity-based chemoproteomics, comprising:
providing a chemoprobe, wherein the chemoprobe is substrate-competitive and selectively binds to an enzymatically active enzyme associated with a gene-specific chromatin regulatory protein complex, wherein the enzymatically active enzyme is present in a functional chromatin regulatory protein complex that is associated with a disease state;
contacting one or more samples with the chemoprobe to screen for the presence of a functional chromatin regulatory protein complex comprising the enzymatically active enzyme in the one or more samples, whereby the functional chromatin regulatory protein complex is isolated from samples where it is present; and
identifying one or more components of the isolated functional chromatin regulatory protein complex associated with the enzymatically active enzyme, whereby an identified component of the isolated functional chromatin regulatory protein complex associated with the enzymatically active enzyme comprises a biomarker for a disease state.

25. The method of claim 24, further comprising defining an architecture of the isolated functional chromatin regulatory protein complex, wherein the defined architecture of the functional chromatin regulatory protein complex comprises a biomarker for disease state.

26. The method of claim 25, further comprising identifying a profile of interacting components within the isolated functional chromatin regulatory protein complex, wherein a defined profile of interacting components of the functional chromatin regulatory protein complex comprises a biomarker for disease state.

27. The method of claim 26, further comprising identifying a co-regulator network.

28. The method of claim 24, wherein the biomarker is for a chronic inflammation-associated disease, optionally wherein the chronic inflammation-associated disease comprises a cancer.

29. The method of claim 24, wherein the contacting one or more samples with the chemoprobe further comprises contacting a first sample from a first subject and contacting a second sample from a second patient, optionally wherein the first subject is a healthy subject and the second subject has a disease phenotype.

30. A component, architecture or profile of components of a functional chromatin regulatory protein complex produced by a method of claim 24.
